SEQUENCE LISTING (1 sequence)

SEQ ID NO: 1 - protein, 14 aa, synthetic construct
  REGION 1..14: note = Enzymatically produced peptide
PKSCDKTHTCPPCP

(19) United States
(12) Patent Application Publication
(10) Pub. No.: US 2023/0043610 A1
(43) Pub. Date: Feb. 9, 2023

(54) NATIVE MICROFLUIDIC CE-MS ANALYSIS OF ANTIBODY CHARGE HETEROGENEITY

(71) Applicant: Regeneron Pharmaceuticals, Inc., Tarrytown, NY (US)

(72) Inventors: Hongxia Wang, Briarcliff Manor, NY (US); Haibo Qiu, Hartsdale, NY (US); Ning Li, New Canaan, CT (US); Zhijie Wu, Stamford, CT (US)

(21) Appl. No.: 17/870,368

(22) Filed: Jul. 21, 2022

Related U.S. Application Data
(63) Continuation-in-part of application No. 16/777,230, filed on Jan. 30, 2020.
(60) Provisional application No. 62/799,331, filed on Jan. 31, 2019.
(60) Provisional application No. 62/851,365, filed on May 22, 2019.

(51) Int. Cl. G01N 33/68; G01N 1/40

(52) U.S. Cl. CPC G01N 33/6848; G01N 1/4044

(57) ABSTRACT

The present invention pertains to methods for characterizing proteins in a sample using native capillary electrophoresis-mass spectrometry. The present invention pertains to methods for detecting and/or discriminating between post-translational modification variants of an antibody of interest in a sample, detecting and/or discriminating between antibodies in an antibody mixture, and characterizing monospecific antibody side products in a bispecific antibody sample.

### CROSS-REFERENCE TO RELATED APPLICATIONS

This application is a continuation-in-part of U.S. patent application Ser. No. 16/777,230, filed on Jan. 30, 2020, which claims the benefit of U.S. Provisional Application No. 62/851,365, filed May 22, 2019, and U.S. Provisional Application No. 62/799,331, filed Jan. 31, 2019, each of which is incorporated by reference in its entirety.

### REFERENCE TO A SEQUENCE LISTING

The instant application contains a Sequence Listing which has been filed electronically in XML format and is hereby incorporated by reference in its entirety. Said XML copy, created on Oct. 14, 2022, is named 070816-01839_SL.xml and is 2.27 bytes in size

### FIELD OF THE INVENTION

The present invention pertains to biopharmaceuticals, and relates to the use of capillary electrophoresis and mass spectral analysis to characterize size and charge variants of therapeutic antibodies.

### BACKGROUND

Therapeutic antibodies are a significant class of biotherapeutic products, and they have achieved outstanding success in treating many life-threatening and chronic diseases. However, in certain circumstances therapeutic antibodies, such as monoclonal antibodies (mAbs), are heterogeneous molecules produced in mammalian cells with many product variants, including variants resulting from post-translational modifications (PTMs). Variants produced via PTMs can occur throughout the lifespan of a mAb during production, purification, storage, and post-administration. These variants or product-related modifications are also referred to as product quality attributes (PQAs). Controlling PQAs within predefined acceptance criteria is vital to the biopharmaceutical industry because it ensures consistent product quality and reduces potential impacts on drug safety and efficacy.

Each individual monoclonal antibody may therefore present a unique profile, a characteristic which needs to be taken into consideration during the evaluation of these products both during development and manufacturing of final product. A Food and Drug Administration guidance for industry recommends that sponsors should evaluate susceptibilities of therapeutic proteins to modifications within the in vivo milieu (see, Guidance for Industry, Immunogenicity Assessment for Therapeutic Protein Products. 2014). As a result, in vitro and/or in vivo behavior of many PQAs, including deamidation (see, for example, Huang et al., Analytical chemistry 2005; 77:1432-9; Ouellette et al., mAbs 2013; 5:432-44; Yin et al., Pharmaceutical research 2013; 30:167-78; Li et al., mAbs 2016:0; Li et al., mAbs 2016:0), oxidation (see, for example, Yin et al., Pharmaceutical research 2013; 30:167-78; Li et al., mAbs 2016:0; Li et al., mAbs 2016:0), glycation (see, for example, Goetze et al., Glycobiology 2012; 22:221-34), glycosylation (see, for example, Li et al., mAbs 2016:0; Li et al., mAbs 2016:0; Goetze et al., Glycobiology 2011; 21:949-59; Alessandri et al., mAbs 2012; 4:509-20), disulfides (see, for example, Li Y et al., mAbs 2016:0; Liu et al., The Journal of biological chemistry 2008; 283:29266-72), N-terminal pyroglutamate (see, for example, Yin et al., Pharmaceutical research 2013; 30:167-78; Li et al., mAbs 2016:0; Li et al., mAbs 2016:0; Liu et al., The Journal of biological chemistry 2011; 286:11211-7), and C-terminal lysine removal (see, for example, Li et al., mAbs 2016:0; Cai et al., Biotechnology and bioengineering 2011; 108:404-12) have been investigated in animal or human samples. Accordingly, additional methods of monitoring mAb preparations are needed.

### SUMMARY OF THE INVENTION

A method has been developed for analysis of protein variants, for example size and charge variants of a therapeutic antibody. The method comprises the use of native microfluidic capillary electrophoresis connected in line with mass spectrometry to separate, identify and/or quantify polypeptides in a sample. The method may be used, for example, to characterize post-translational modifications (PTMs) of an antibody, to detect monospecific mAb impurities (referred as either HC/HC or HC*/HC*) in a bispecific antibody (bsAb) sample, and to characterize alternative antibody formats such as “N−1,” which is a one arm antibody, and “N+1” antibodies, where an additional Fab arm was connected to the heavy chain.

In one aspect, the present invention provides a method for detecting and/or discriminating between post-translational modification variants of an antibody of interest in a sample, in which the method includes: contacting a sample comprising one or more antibodies of interest with a protease to digest the sample into antibody fragments; separating antibody fragments by molecular weight and/or charge in one or more capillaries using capillary electrophoresis; eluting separated antibody fragments from the one or more capillaries; and determining the mass of the eluted antibody fragments by mass spec analysis, thereby detecting and/or discriminating between post-translational modification variants of the antibody of interest.

In various embodiments of the method, the post-translational modification comprises one or more of deamidation, oxidation, glycation, disulfide formation, N-terminal pyroglutamate formation, C-terminal lysine removal, high mannose glycosylation, and O-glycosylation.

In various embodiments of the method, the protease comprises IdeS.

In various embodiments of the method, the antibody fragments comprise one or more of an F(ab′)₂ or Fc antibody subunit.

In various embodiments of the method, the antibody of interest is a mAb.

In various embodiments of the method, the antibody fragments are separated by charge and the method is a method of detecting and/or discriminating between charge variants of the antibody of interest.

In various embodiments of the method, the antibody fragments are separated by molecular weight and the method is a method of detecting and/or discriminating between size variants of the antibody of interest.

In some embodiments, the method further includes determining a relative or absolute amount of the post-translational modification variants of an antibody of interest in a sample.

In various embodiments of the method, the antibody of interest comprises a bispecific antibody.

In various embodiments of the method, the sample includes an internal standard.

In various embodiments of the method, the one or more capillaries comprise a separation matrix.

In various embodiments of the method, the separation matrix comprises a sieving matrix configured to separate proteins by molecular weight.

In various embodiments of the method, eluting separated antibody fragments from the one or more capillaries further comprises separating the antibody fragments into one or more fractions.

In some embodiments, the method further includes identifying the antibody fragments.

In some embodiments, the method further includes identifying the post-translational modification present on the antibody fragments.

In various embodiments of the method, the monoclonal antibody of interest is of isotype IgG1, IgG2, IgG3, IgG4, or mixed isotype.

In some embodiments, the method further includes post-translational modification profiling of the antibody of interest.

In some embodiments, the method further includes post-translational modification mapping of post-translational modification hotspots by reduced peptide mapping LC-MS/MS analysis.

In various embodiments of the method, the sample comprises a mixture of antibodies of interest.

In various embodiments of the method, the monoclonal antibody of interest is an antibody drug conjugate.

This disclosure provides a further method for detecting and/or discriminating between post-translational modification variants of an antibody of interest in a sample. In some exemplary embodiments, the method comprises (a) contacting a sample comprising one or more antibodies of interest with a protease to digest the sample into antibody fragments; (b) separating antibody fragments by molecular weight and/or charge in one or more capillaries using capillary electrophoresis; (c) eluting separated antibody fragments from the one or more capillaries; and (d) determining the mass of the eluted antibody fragments by mass spectrometry analysis, thereby detecting and/or discriminating between post-translational modification variants of the antibody of interest, wherein said one or more antibodies of interest are maintained in native conditions, and wherein said capillary electrophoresis is in an integrated microfluidic platform.

In one aspect, the post-translational modification comprises one or more of deamidation, oxidation, glycation, disulfide formation, N-terminal pyroglutamate formation, C-terminal lysine removal, high mannose glycosylation, and O-glycosylation.

In one aspect, the protease comprises IdeS.

In one aspect, the antibody fragments comprise one or more of an F(ab′)₂ or Fc antibody subunit.

In one aspect, the antibody of interest is a monoclonal antibody.

In one aspect, the antibody fragments are separated by charge and the method is a method of detecting and/or discriminating between charge variants of the antibody of interest.

In one aspect, the antibody fragments are separated by molecular weight and the method is a method of detecting and/or discriminating between size variants of the antibody of interest.

In one aspect, the method further comprises determining a relative or absolute amount of the post-translational modification variants of an antibody of interest in a sample.

In one aspect, the antibody of interest comprises a bispecific antibody.

In one aspect, the sample includes an internal standard.

In one aspect, the one or more capillaries comprise a separation matrix. In a specific aspect, said separating comprises a sieving matrix configured to separate proteins by molecular weight.

In one aspect, eluting separated antibody fragments from said one or more capillaries further comprises separating the antibody fragments into one or more fractions.

In one aspect, the method further comprises identifying the antibody fragments.

In one aspect, the method further comprises identifying the post-translational modification present on the antibody fragments.

In one aspect, the antibody of interest is of isotype IgG1, IgG2, IgG3, IgG4, or mixed isotype.

In one aspect, the method further comprises post-translational modification profiling of the antibody of interest.

In one aspect, the method further comprises post-translational modification mapping of post-translational modification hotspots by reduced peptide mapping LC-MS/MS analysis.

In one aspect, the sample comprises a mixture of antibodies of interest.

In one aspect, the antibody of interest is an antibody drug conjugate. In another aspect, the antibody of interest is a “N−1” antibody or a “N+1” antibody.

In one aspect, an injection volume in the one or more capillaries is between about 1 nL and about 10 nL. In a specific aspect, an injection volume in the one or more capillaries is about 1 nL.

This disclosure provides a further method for detecting and/or discriminating between post-translational modification variants of an antibody of interest in a sample. In some exemplary embodiments, the method comprises (a) contacting a sample comprising one or more antibodies of interest with an IdeS protease to digest the sample into antibody fragments, wherein the protease to sample ratio is about 1.25 units of protease to about 1 μg sample; (b) separating antibody fragments by molecular weight and/or charge in one or more capillaries comprising a sieving matrix using capillary electrophoresis; (c) eluting separated antibody fragments from the one or more capillaries; and (d) determining the mass of the eluted antibody fragments by mass spectrometry analysis, thereby detecting and/or discriminating between post-translational modification variants of the antibody of interest, wherein said antibody of interest is maintained in native conditions, and wherein said capillary electrophoresis is in an integrated microfluidic platform.

This disclosure also provides a method for characterizing a monospecific antibody in a mixture of a bispecific antibody and its monospecific antibody side products. In some exemplary embodiments, the method comprises (a) separating a mixture of a bispecific antibody and its monospecific antibody side products by molecular weight and/or charge in one or more capillaries using capillary electrophoresis; (b) eluting said separated antibody and antibody side products from said one or more capillaries; and (c) determining the mass of said eluted antibody and antibody side products by mass spectrometry, thereby characterizing said monospecific antibody, wherein said monospecific antibody is maintained in native conditions, and wherein said capillary electrophoresis is in an integrated microfluidic platform.

In one aspect, the method further comprises determining a relative or absolute amount of the monospecific antibody in said mixture.

In one aspect, the mixture includes an internal standard.

In one aspect, the one or more capillaries comprise a separation matrix. In a specific aspect, the separation matrix comprises a sieving matrix configured to separate proteins by molecular weight.

In one aspect, the monospecific antibody is of isotype IgG1, IgG2, IgG3, IgG4, or mixed isotype.

In one aspect, the method further comprises characterizing a second monospecific antibody in the mixture.

In one aspect, an injection volume in the one or more capillaries is between about 1 nL and about 10 nL. In a specific aspect, an injection volume in the one or more capillaries is about 1 nL.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### BRIEF DESCRIPTION OF THE DRAWINGS

FIG. 1 illustrates an exemplary work flow for the separation and detection of post-translational modified antibody fragments by capillary electrophoresis and mass spectral analysis, according to an exemplary embodiment.

FIG. 2A shows electropherograms evaluating the sensitivity of ZipChip nCE-MS for NISTmAb (IgG1), according to an exemplary embodiment.

FIG. 2B shows electropherograms evaluating the sensitivity of ZipChip nCE-MS for mAb1 (IgG1), according to an exemplary embodiment.

FIG. 2C shows electropherograms evaluating the sensitivity of ZipChip nCE-MS for mAb2 (IgG4), according to an exemplary embodiment.

FIG. 2D shows electropherograms evaluating the sensitivity of ZipChip nCE-MS for bsAb1 (IgG4), according to an exemplary embodiment.

FIG. 3 shows mass spectra at the limit of detection (LOD) for NISTmAb, mAb1, mAb2, and bsAb1, according to an exemplary embodiment.

FIG. 4A shows sensitivity of ZipChip nCE-MS for IgG1, according to an exemplary embodiment.

FIG. 4B shows sensitivity of ZipChip nCE-MS for IgG4, according to an exemplary embodiment.

FIG. 4C shows a carryover test of ZipChip nCE-MS, according to an exemplary embodiment.

FIG. 5A shows a charge variant profile of deglycosylated NISTmAb, including two basic variants and two acidic variants, according to an exemplary embodiment.

FIG. 5B shows a mass spectrum of the acidic 2 variant of NISTmAb, demonstrating Fab glycosylation, according to an exemplary embodiment.

FIG. 5C shows a zoom-in view of a deglycosylated NISTmAb electropherogram, showing three truncated forms, according to an exemplary embodiment.

FIG. 5D shows mass spectra of three NISTmAb truncation forms cleaved at (i) Cys223/Asp224, (ii) Lys225/Thr226, and (iii) His227/Thr228, according to an exemplary embodiment.

FIG. 6 shows the charge variant separation of intact NISTmAb and SEQ ID NO: 1, according to an exemplary embodiment.

FIG. 7A shows an electropherogram of charge variant analysis of antibody F(ab′)2 and Fc subunits for IdeS treated control and stressed NISTmAb, according to an exemplary embodiment.

FIG. 7B shows an electropherogram of charge variant analysis of antibody F(ab′)2 subunits for IdeS treated control and stressed NISTmAb, according to an exemplary embodiment.

FIG. 7C shows an electropherogram of charge variant analysis of antibody Fc subunits for IdeS treated control and stressed NISTmAb, according to an exemplary embodiment.

FIG. 8A shows a separation of three IgG1 mAbs using ZipChip nCE-MS, according to an exemplary embodiment.

FIG. 8B shows a separation of five bispecific IgG4 mAbs using ZipChip nCE-MS, according to an exemplary embodiment.

FIG. 8C shows a separation of ten mAbs using ZipChip nCE-MS, according to an exemplary embodiment.

FIG. 8D shows an identification of co-migrated mAbs using ZipChip nCE-MS, according to an exemplary embodiment.

FIG. 8E shows an identification of co-migrated mAbs using ZipChip nCE-MS, according to an exemplary embodiment.

FIG. 9 shows electropherograms for five lots of formulated antibodies manufactured from two different processes, according to an exemplary embodiment.

FIG. 10 shows mass spectra of main species and basic variant 1 for five lots of mAb3 manufactured with two different processes, according to an exemplary embodiment.

FIG. 11A shows charge variant separation by SCX-UV, according to an exemplary embodiment.

FIG. 11B shows charge variant separation by ZipChip nCE-MS, according to an exemplary embodiment.

FIG. 12A shows charge variant separation by iCIEF, according to an exemplary embodiment.

FIG. 12B shows charge variant separation by ZipChip nCE-MS, according to an exemplary embodiment.

FIG. 13A shows an electropherogram of bsAb1 with 20 ng injection by ZipChip nCE-MS, according to an exemplary embodiment.

FIG. 13B shows a 10× zoom-in of an electropherogram of bsAb1 with 20 ng injection by ZipChip nCE-MS, according to an exemplary embodiment.

FIG. 13C shows HC*/HC* monospecific mAb impurities at spike-in levels of 1:500 and 1:100 in bsAb1 by ZipChip nCE-MS, according to an exemplary embodiment.

FIG. 13D shows a zoom-in view of HC*/HC* monospecific mAb impurities at spike-in levels of 1:500 and 1:100 in bsAb1 by ZipChip nCE-MS, according to an exemplary embodiment.

FIG. 14 shows mass spectra for HC/HC in a bsAb1 sample, HC/HC standard, and half mAb by ZipChip nCE-MS, according to an exemplary embodiment.

FIG. 15 shows mass spectra of HC*/HC* at 0.2% and 1% using ZipChip nCE-MS, according to an exemplary embodiment.

FIG. 16A shows an electropherogram of an “N−1” format antibody using ZipChip nCE-MS, according to exemplary embodiment.

FIG. 16B shows an electropherogram of an “N+1” format antibody using ZipChip nCE-MS, according to an exemplary embodiment.

FIG. 17 shows identification of O-glycosylation (Xyl+Gal+Gal+GlcA) in acidic variant 1 of mAb7 using ZipChip nCE-MS, according to an exemplary embodiment.

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

Abbreviations Used Herein

mAb: Monoclonal antibody

biAb/bsAb: Bispecific antibody

CQA: Critical quality attributes

CE: Capillary Electrophoresis

PTM: Post-Translational Modification

IEC: Ion Exchange Chromatography

UV: Ultra Violet

QC: Quality Control

MS: Mass Spectrometry

ADC: Antibody Drug Conjugate

The term “antibody”, as used herein, is intended to refer to immunoglobulin molecules included of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is included of a heavy chain variable region (“HCVR” or “V_{H}”) and a heavy chain constant region (included of domains C_{H}1, C_{H}2 and C_{H}3). In various embodiments, the heavy chain may be an IgG isotype. In some cases, the heavy chain is selected from IgG1, IgG2, IgG3 or IgG4. In some embodiments, the heavy chain is of isotype IgG1 or IgG4, optionally including a chimeric hinge region of isotype IgG1/IgG2 or IgG4/IgG2. Each light chain is included of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. For a review on antibody structure, see Lefranc et al., IMGT unique numbering for immunoglobulin and T cell receptor variable domains and Ig superfamily V-like domains, 27(1) Dev. Comp. Immunol. 55-77 (2003); and M. Potter, Structural correlates of immunoglobulin diversity, 2(1) Surv. Immunol. Res. 27-42 (1983).

The term antibody also encompasses a “bispecific antibody”, which includes a heterotetrameric immunoglobulin that can bind to more than one different epitope. One half of the bispecific antibody, which includes a single heavy chain and a single light chain and six CDRs, binds to one antigen or epitope, and the other half of the antibody binds to a different antigen or epitope. In some cases, the bispecific antibody can bind the same antigen, but at different epitopes or non-overlapping epitopes. In some cases, both halves of the bispecific antibody have identical light chains while retaining dual specificity. Bispecific antibodies are described generally in U.S. Patent App. Pub. No. 2010/0331527 (Dec. 30, 2010).

Co-expression of two unique heavy chains (HC and HC*) and one common light chain will minimize a number of side products to two monospecific mAb impurities, which may need to be subsequently detected and removed during purification. The two impurities may be referred to as HC/HC and HC*/HC* respectively. In some exemplary embodiments, the CH3 domain of one heavy chain contains amino acid substitutions that prevent binding to protein A, for example H95R and Y96F substitutions. In some exemplary embodiments, the method of the present invention can be used to detect, identify, quantify, and/or remove monospecific mAb impurities in a sample comprising a bispecific antibody.

The term antibody also encompasses alternative antibody formats including, for example, “N−1”, which is a one arm antibody, and “N+1”, where an additional Fab arm is connected using, for example, a G4S linker. The method of the present invention can also be used in the analysis of, for example, antibody subunits, antibody fusion proteins, single chain variable fragments (scFvs), diabodies, triabodies, and other antigen-binding proteins or any protein of interest.

The term “antigen-binding portion” of an antibody (or “antibody fragment”), refers to one or more fragments of an antibody that retain the ability to specifically bind to an antigen. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et at. (1993) 90 PNAS U.S.A. 6444-6448; and Poljak et at. (1994) 2 Structure 1121-1123).

Moreover, antibodies and antigen-binding fragments thereof can be obtained using standard recombinant DNA techniques commonly known in the art (see Sambrook et al., 1989).

The term “human antibody”, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “ADC” or “antibody-drug conjugate” refers to an antibody or antigen-binding fragment thereof conjugated to a therapeutic moiety such as a cytotoxic agent, a chemotherapeutic drug, immunosuppressant or a radioisotope. Cytotoxic agents include any agent that is detrimental to the growth, viability or propagation of cells. Examples of suitable cytotoxic agents and chemotherapeutic agents for forming ADCs are known in the art.

The term “sample,” as used herein, refers to a mixture of molecules that includes at least one polypeptide of interest, such as a monoclonal antibody or a bispecific antibody or fragment thereof, that is subjected to manipulation in accordance with the methods of the invention, including, for example, separating, analyzing, extracting, concentrating or profiling.

The terms “analysis” or “analyzing,” as used herein, are used interchangeably and refer to any of the various methods of separating, detecting, isolating, purifying, solubilizing, detecting and/or characterizing molecules of interest (e.g., polypeptides, such as antibodies) and contaminants in antibody preparations. Examples include, but are not limited to, electrophoresis, mass spectrometry, e.g. tandem mass spectrometry, ultraviolet detection, and combinations thereof.

“Chromatography,” as used herein, refers to the process of separating a mixture, for example a mixture containing peptides, proteins, polypeptides and/or antibodies, such as monoclonal antibodies. It involves passing a mixture through a stationary phase, which separates molecules of interest from other molecules in the mixture and allows one or more molecules of interest to be isolated.

The term “isolated,” as used herein, refers to a biological component (such as an antibody, for example a monoclonal antibody) that has been substantially separated, produced apart from, or purified away from other biological components in the cell of the organism in which the component naturally occurs or is transgenically expressed, that is, other chromosomal and extrachromosomal DNA and RNA, proteins, lipids, and metabolites. Nucleic acids, peptides, proteins, lipids and metabolites which have been “isolated” thus include nucleic acids, peptides, proteins, lipids, and metabolites purified by standard or non-standard purification methods. The term also embraces nucleic acids, peptides, proteins, lipids, and metabolites prepared by recombinant expression in a host cell as well as chemically synthesized peptides, lipids, metabolites, and nucleic acids.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan W Trp; and Tyrosine Y Tyr. In one embodiment, a peptide is an antibody or fragment or part thereof, for example, any of the fragments or antibody chains listed above. In some embodiments, the peptide may be post-translationally modified. As used herein, the terms “protein of interest” and/or “target protein of interest” refer to any protein to be separated and/or detected with the methods provided herein. Suitable proteins of interest include antibodies, for example monoclonal antibodies, and fragments thereof.

“Detect” and “detection” have their standard meaning, and are intended to encompass detection including the presence or absence, measurement, and/or characterization of a protein of interest, such as a mAb or fragment thereof.

As used herein, the terms “standard” and/or “internal standard” refer to a well-characterized substance of known amount and/or identity (e.g., known molecular weight, electrophoretic mobility profile) that can be added to a sample and both the standard and the molecules in the sample, on the basis of molecular weight or isoelectric point by electrophoresis). A comparison of the standard then provides a quantitative or semi-quantitative measure of the amount of analyte, such as mAb or fragments thereof present in the sample.

“Contacting,” as used herein, includes bringing together at least two substances in solution or solid phase, for example contacting a sample with an enzyme, such as a protease.

The term “corresponding” is a relative term indicating similarity in position, purpose or structure, and may include peptides of identical structure but for the presence or absence of a post-translational modification. In some embodiments, mass spectral signals in a mass spectrum that are due to corresponding peptides of identical structure but for the presence or absence of a post-translational modification are “corresponding” mass spectral signals. A mass spectral signal due to a particular peptide is also referred to as a signal corresponding to the peptide. In certain embodiments, a particular peptide sequence or set of amino acids can be assigned to a corresponding peptide mass.

The terms “fragment peptide” or “peptide fragment,” as used herein, refer to a peptide that is derived from the full-length polypeptide, such as a protein and/or monoclonal antibody, through processes including fragmentation, enzymatic proteolysis, or chemical hydrolysis. Such proteolytic peptides include peptides produced by treatment of a protein with one or more proteases, such as IdeS protease. A fragment peptide, or peptide fragment, can be a digested peptide.

“Mass spectrometry” refers to a method in which a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), orbitrap mass analyzer, Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography.

Tandem mass spectrometry or MS/MS is a technique to break down selected ions (precursor ions) into fragments (product ions). The fragments then reveal aspects of the chemical structure of the precursor ion. In tandem mass spectrometry, once samples are ionized (for example by ESI, MALDI, EI, etc.) to generate a mixture of ions, precursor ions, for example peptides from a digest of a specific mass-to-charge ratio (m/z) are selected (MS1) and then fragmented (MS2) to generate product ions for detection. Typical tandem MS instruments include QqQ, QTOF, and hybrid ion trap/FTMS, etc. One example of an application of tandem mass spectrometry is protein identification. The first mass analyzer isolates ions of a particular m/z value that represent a single species of peptide among many introduced into and then emerging from the ion source. Those ions are then accelerated into a collision cell containing an inert gas such as argon to induce ion fragmentation. This process is designated collisionally induced dissociation (CID) or collisionally activated dissociation (CAD). The m/z values of fragment ions are then measured in a 2^{nd} mass analyzer to obtain amino acid sequence information.

References to a mass of an amino acid mean the monoisotopic mass or average mass of an amino acid at a given isotopic abundance, such as a natural abundance. In some examples, the mass of an amino acid can be skewed, for example, by labeling an amino acid with an isotope. Some degree of variability around the average mass of an amino acid is expected for individual single amino acids based on the exact isotopic composition of the amino acid. The masses, including monoisotopic and average masses for amino acids are easily obtainable by one of ordinary skill the art.

Similarly, references to a mass of a peptide means the monoisotopic mass or average mass of a peptide at a given isotopic abundance, such as a natural abundance. In some examples, the mass of a peptide can be skewed, for example, by labeling one or more amino acids in the peptide with an isotope. Some degree of variability around the average mass of a peptide is expected for individual single peptides based on the exact isotopic composition of the peptide. The mass of a particular peptide can be determined by one of ordinary skill the art.

In some exemplary embodiments, CE, MS, and CE-MS can be performed under native conditions (nCE-MS). As used herein, the term “native conditions” can include performing capillary electrophoresis and/or mass spectrometry under conditions that preserve non-covalent interactions in an analyte, in contrast to, for example, reducing and/or denaturing conditions. Native capillary electrophoresis and/or mass spectrometry is an approach to study intact biomolecular structure in the native or near-native state. The term “native” refers to the biological status of the analyte in solution prior to subjecting to separation and/or ionization. Several parameters, such as pH and ionic strength, of the solution containing the biological analytes can be controlled to maintain the native folded state of the biological analytes in solution.

General Description

Therapeutic antibodies are a major class of biopharmaceuticals that have been developed as treatments for a variety of therapeutic areas including infectious diseases (Sparrow et al., 2017, Bull. World Health Organ., 95(3):235-237), inflammation and immunology (Chan et al., 2010, Nat. Rev. Immunol., 10(50):301-316), oncology (Scott et al., 2012, Nat. Rev. Cancer., 12(4):278-287), hematology (Cuesta-Mateos et al., 2017, Front. Immunol., 8:1936), ophthalmology (Rodrigues et al., 2009, Prog. Retin. Eye Res.), 28(2):117-144, and rare diseases (Tambuyzer et al., 2020, Nat. Rev. Drug Discov., 19(2):93-111). The advantages of therapeutic antibodies include the high specificity and binding affinity to their molecular targets, and generally low immunotoxicity and mild adverse effects (Rodrigues et al.; Lu et al., 2020, J. Biomed. Sci., 27(1):1). Monoclonal antibodies (mAbs) including immunoglobulin G (IgG) subclasses, such as IgG1 and IgG4, have attracted the most attention, because of their simpler structures and longer half-lives than those of other subclasses. The structure of these antibodies consists of two identical heavy chains and light chains, with intrachain and interchain disulfide bond linkage (Chiu et al., 2019, Antibodies (Basel), 8:55). Additionally, bispecific antibodies (bsAbs), in which the two heavy chains and light chains may be different, and other IgG-based molecules in alternative formats, such as those with addition and removal of the antigen-binding fragment (Fab) arm, have also been created to achieve advantageous properties such as the ability to interact with multiple therapeutic targets (Labrijn et al., 2019, Nat. Rev. Drug Discov., 18(8):585-608; Seung et al., 2022, Nature, 603(7900):328-334; Spiess et al., 2015, Mol. Immunol., 67(95-106)). In cancer treatment, bsAbs can achieve high target specificity through one paratope and recruitment of tumor killing agents through the other paratope (Runcie et al., 2018, Mol. Med., 24(1):50).

Characterization of antibody variants is important in order to identify their potential impact on safety, potency, and stability of a potential therapeutic antibody. For example, to be considered for approval by regulatory agencies, extensive characterization of the molecule must be performed. In drug products comprising mixtures of antibodies, characterization of the absolute or relative amounts of each antibody must be determined. Because aggregates and fragments may potentially affect immunogenicity and potency, their levels are typically monitored during lot release, stability, and characterization. Furthermore, primary degradation pathways for the molecule and product related impurities and variants are determined.

Characterization of these therapeutic antibodies and their derivatives has become more challenging, owing to the increasing complexity of these biomolecules and their heterogeneity. For example, post-translational modifications (PTMs) commonly occur on therapeutic antibodies in bioreactors during production (Wurm, 2004, Nat. Biotechnol., 22(11):1393-1398). These modifications include glycosylation, asparagine deamidation, aspartic acid isomerization and cyclization, methionine and tryptophan oxidation, and lysine glycation, among others (Liu et al., 2008, J. Pharm. Sci., 97(7):2426-2447). As amino acid residues are modified, the electrostatic environment surrounding the amino acids changes, thereby resulting in a net charge difference in the molecule and the formation of charge variants (Du et al., 2012, MAbs, 4(5):578-585). For instance, lysine glycation adds a neutrally charged monosaccharide to a positively charged and basic lysine residue, thus resulting in an acidic variant. Methionine oxidation changes a methionine residue to a methionine sulfone, thus resulting in a basic variant. Moreover, cellular processing also causes the formation of charge variants, including incomplete cleavage of C-terminal lysine, and incomplete cyclization of the N-terminal glutamine residue. Mass spectrometry (MS) has been extensively used to study PTMs at the peptide, subunit, and intact levels (Zhang et al., 2014, FEBS Lett., 588(2):308-317). Peptide mapping, wherein therapeutic antibodies are digested with enzymes, can be used to monitor multiple PTMs simultaneously and have been widely adopted (Rogstad et al., 2017, J. Am. Soc. Mass Spectrom., 28(5):786-794). Additionally, intact mass analysis of therapeutic antibodies at both the subunit and intact levels has also been performed (Jin et al., 2019, MAbs, 11(1): 106-115).

Charge variant analysis is critical for understanding the structure of biotherapeutics during drug development and product release (Du et al.). The charge variant profile can be characterized by several analytical techniques, including imaged capillary isoelectric focusing (iCIEF) and cation exchange chromatography (CEX), to enable relative quantification of charge variants. The iCIEF technique separates charge species according to the isoelectric point (pI) (Salas-Solano et al., 2011, Chromatographia, 73(11):1137-1144). CEX is a chromatographic technique that separates species according to surface charge, and strong cation exchange (SCX) is a common method used for therapeutic antibody analysis (Fekete et al., 2015, J. Pharm. Biomed. Anal., 113:43-55). For iCIEF, electropherograms are generated according to optical detection, and thus the identity of the separated charge variants is not determined. Although several iCIEF-MS platforms have been demonstrated to provide pI based separation and charge variant identification (Mack et al., 2019, Electrophoresis, 40(23-24):3084-3091; He et al., 2022, Electrophoresis), these platforms remain under development and are not widely adopted. Recent advances have enabled direct coupling of CEX to MS through use of MS-compatible salt; however, higher resolution separation is normally achieved with MS-incompatible buffer rather than MS-compatible buffer (Yan et al., 2018, Anal. Chem., 90(21):13013-13020; Ma et al., 2020, MAbs, 12(1):1763762).

ZipChip is an integrated microfluidic capillary electrophoresis (CE) system that can be directly coupled with the MS interface, and has been used to analyze small molecules in media, monosaccharides and oligosaccharides (Ribeiro da Silva et al., 2021, J. Chromatogr. A, 1651:462336; Khatri et al., 2017, Anal. Chem., 89(12):6645-6655); peptides for antibody protein sequence confirmation and proteomic analysis (Khatri et al.; Dykstra et al., 2021, J. Am. Soc. Mass Spectrom., 32(8): 1952-1963; Cao et al., 2021, J. Pharm. Biomed. Anal., 204:114251); and intact proteins including fusion proteins (Deyanova et al. 2021, Electrophoresis, 42(4):460-464), mAbs (Cao et al.; Sun et al., 2021, Anal. Biochem., 625:114214; Redman et al., 2015, Anal. Chem., 87(4):2264-2272), and antibody-drug conjugates (Redman et al., 2016, Anal. Chem., 88(4):2220-2226). Due to the low flow nature of CE and high ionization efficiency of nano-electrospray, this integrated system is able to provide high resolution separation and high sensitivity detection. With the native CE-MS (nCE-MS) method, charge variants are separated on the basis of both protein charge and size under native conditions (Redman et al., 2015). During separation, the charge variants with more positive charges migrate faster and elute earlier in the native BGE (pH<pI of antibody), whereas variants with fewer positive charges migrate slowly in the separation channel under the electric field. Therefore, in a typical antibody charge separation profile, basic variants elute first, followed by the main species and acid variants. Additionally, the ZipChip interface is compatible with common formulation buffer components containing anionic or nonionic detergents for easy sample preparation.

Disclosed herein is a highly sensitive, high-resolution microfluidic nCE-MS method for detecting and/or discriminating between variants of an antibody of interest, such as a therapeutic antibody, in a sample by a physical parameter, such as mass and/or charge. The method may be used, for example, for antibody charge variant and impurity analysis, and for the quick identification of antibody charge variants for forced degradation and long-term stability studies. The sensitivity was first evaluated with three types of therapeutic antibodies. Additionally, antibody samples from different manufacturing processes and lots, as well as the antibody cocktail, were analyzed with the nCE-MS method. Comparative analysis of charge variant profiles was performed for the nCE-MS, SCX and iCIEF methods. The high sensitivity of the nCE-MS method enabled detection of fragments with very low abundance in the antibody sample and homospecific mAb impurities in the bsAb sample. Finally, new antibody modalities were evaluated with the nCE-MS method.

The disclosed methods can be used in QC evaluation of antibody preparations. In embodiments of the method, a sample that includes an antibody of interest is resolved or separated by using capillary electrophoresis, for example on one or more capillaries of a CE-system. In certain embodiments, the sample is resolved or separated by molecular weight and charge. For example, using separation by mass and charge, or m/z ratio, fragments with the same mass but different charges can be resolved. Similarly, using separation by mass and charge, or m/z ratio, fragments with the same charge but different masses can be resolved. In embodiments, the method includes liberating fragments of an antibody of interest, such as a monoclonal antibody (mAb), for example by contacting the sample comprising one or more antibodies of interest with a protease to digest the sample. In an embodiment, the protease is IdeS protease. Once digestion, either partial or full, is conducted, antibody fragments can be separated by molecular weight and/or charge in one or more capillaries using capillary electrophoresis. The separated antibody fragments can be eluted from the one or more capillaries and the mass of the eluted antibody fragments determined by mass spec analysis to detect and/or discriminate between post-translational modification variants of the antibody of interest, for example by detection and/or determination of the PTM profile of the fragments of the antibody of interest. In certain embodiments, the antibody fragments include one or more of an F(ab′)₂ or Fc antibody subunit, for example as digested from the intact antibody using a protease, such as the IdeS protease. In certain embodiments, the antibody of interest is a monoclonal antibody, such as a currently used therapeutic antibody or one undergoing evaluation, including novel monoclonal antibodies. In certain embodiments, the monoclonal antibody of interest is part of an antibody drug conjugate (ADC). In certain embodiments, the antibody fragments are separated by charge and the method is a method of detecting and/or discriminating between charge variants of the antibody of interest. In certain embodiments, the antibody fragments are separated by molecular weight and the method is a method of detecting and/or discriminating between size variants of the antibody of interest. In certain embodiments, the antibody fragments are separated by charge and molecular weight and the method is a method of detecting and/or discriminating between charge and molecular weight variants of the antibody of interest. In certain embodiments, the method includes determining a relative or absolute amount of the post-translational modification variants of an antibody of interest in a sample, for example from the antibody fragments.

As noted above, separation of the antibody fragments by mass and charge has the benefit of being able to determine the homogeneity of the antibody fragments, for example, changes in surface charge of the antibody that may not be easily seen in separation by just molecular weight. This separation allows for the determination of the type and level of post-translational modification on the fragments in the sample. The presence of post-translational modifications (PTMs) on a monoclonal antibody (mAb) induces charge heterogeneity (see Table 1) and potentially affects drug stability and biological activity. Therefore, monitoring the PTMs and associated charge variant profiles of mAbs during drug development is important. Disclosed herein is the development of a high-throughput and highly sensitive native microfluidic CE-MS method for the quick identification of mAb charge variants and its application to forced degradation and long-term stability studies. Relative to ion exchange chromatography (IEX) based approaches, high resolution with comparable charge variant profiles can be obtained using the native microfluidic CE-MS method as disclosed herein.

TABLE 1
Source of antibody charge heterogeneity

| Major PTMs/Degradation | | Species |
|---|---|---|
| Pathway | Effect | Formed |
| Sialylation | COOH addition | Acidic |
| Deamidation | COOH formation | Acidic |
| C-terminal lysine cleavage | Loss of NH2 | Acidic |
| Adduct formation | COOH formation or loss of NH2 | Acidic |
| Succinimide formation | Loss of COOH | Basic |
| Methionine, cysteine, lysine, | Conformational change | Basic |
| histidine, tryptophan oxidation | | |
| Disulfide-mediated | Conformational change | Basic |
| Asialylation (terminal | Loss of COOH | Basic |
| Galactose) | | |
| C-terminal lysine and glycine | NH2 formation or loss of COOH | Basic |
| amidation | | |

In certain embodiments, the post-translational modification is one or more of deamidation, oxidation, glycation, disulfide formation, N-terminal pyroglutamate formation, C-terminal lysine removal, high mannose glycosylation, and O-glycosylation.

In certain embodiments, the sample is resolved or separated within a single capillary. In certain embodiments, the sample is resolved or separated within multiple capillaries, for example in parallel. By way of example with respect to separation by molecular weight, the smaller the fragment of an antibody, the further within a capillary it would be expected to travel over a given period of time. In addition, one would expect differences in the charge of antibody fragments to be subjected to different travel times depending on the charge.

In embodiments, the sample may contain multiple, such as at least 2, at least 3, at least 4, at least 5 or more sets of antibody fragments from multiple antibodies of interest. In some embodiments, the method further includes determining a relative or absolute amount of the variants of the antibody fragments in a sample, for example by measurement of peak height or area, which corresponds to the amount of antibody fragment in the sample. In some embodiments, the antibody of interest comprises a bispecific monoclonal antibody. In some embodiments, the sample includes one or more internal standards, for example a ladder of molecular weight standards, a ladder of isoelectric point standards, or even a standard used as a baseline or benchmark for determining the amount of an antibody fragments of interest in the sample.

The ability to discriminate between mAbs in a mAb cocktail of multiple mAbs is becoming increasingly important as these multiple component therapies demonstrate increased efficacy in disease treatment. Thus, improved methods of monitoring how the individual mAbs behave in these systems will become increasingly important in the assessment of the compatibility and stability of these multi-mAb therapies. To meet this growing need this disclosure provides a method for detecting and/or discriminating between monoclonal antibodies in a mixture of two of more monoclonal antibodies in a sample.

In embodiments, the method includes separating protein components of a sample with two or more mAbs of interest, such as 2, 3, 4, 5, 6, 7, 8, 9 10 or even more, mAbs of interest, by charge in one or more capillaries using capillary electrophoresis

In some embodiments, a charge based profile or fingerprint of the antibody of interest can be created, for example of the antibody of interest alone for comparison with a charge based profile or fingerprint of the antibody in the mixture, for example a charge based profile or fingerprint corresponding to the post-translational modification. This comparison can then be used to determine if the antibody of interest changes in the mixture. This profile or fingerprint comparison can be done for any or all of the antibodies of interest in the mixture.

Samples for use in the disclosed methods can be heterogeneous, containing a variety of components, i.e. different proteins. Alternatively, the sample can be homogenous, containing one component or essentially one component of multiple charge or molecular weight species. Pre-analysis processing may be performed on the sample prior to detecting the antibody of interest, such as a mAb or multiple mAbs. For example, the sample can be subjected to a lysing step, denaturation step, heating step, purification step, precipitation step, immunoprecipitation step, column chromatography step, centrifugation, etc. In some embodiments, the separation of the sample and immobilization may be performed on native substrates. In other embodiments, the sample may be subjected to denaturation, for example, heat and/or contact with a denaturizing agent. Denaturizing agents are known in the art. In some embodiments, the sample may be subjected to non-reducing conditions. In some embodiments, the sample may be subjected to reducing conditions, for example, by contacting the sample with one or more reducing agents. Reducing agents are knowns in the art.

In embodiments, the capillary may include a separation matrix, which can be added in an automated fashion by the apparatus and/or system. In some embodiments, the sample is loaded onto a stacker matrix prior to separation. The separation matrix, in one embodiment, is a size separation matrix, and has similar or substantially the same properties of a polymeric gel, used in conventional electrophoresis techniques. Capillary electrophoresis in the separation matrix is analogous to separation in a polymeric gel, such as a polyacrylamide gel or an agarose gel, where molecules are separated on the basis of the size of the molecules in the sample, by providing a porous passageway through which the molecules can travel. The separation matrix permits the separation of molecules by molecular size because larger molecules will travel more slowly through the matrix than smaller molecules. In some embodiments, the one or more capillaries comprise a separation matrix. In some embodiments, the sample containing an antibody of interest is separated or resolved based on molecular weight. In some embodiments, the separation matrix comprises a sieving matrix configured to separate proteins by molecular weight. In some embodiments, protein components of a sample are separated by molecular weight and the method is a method of detecting and/or discriminating between size variants of an antibody of interest. In some embodiments, antibody fragments of a sample are separated by molecular weight and the method is a method of detecting and/or discriminating between size variants of a contaminating protein of interest.

A wide variety of solid phase substrates are known in the art, for example gels, such as polyacrylamide gel. In some embodiments, resolving one or more proteins of interest includes electrophoresis of a sample in a polymeric gel. Electrophoresis in a polymeric gel, such as a polyacrylamide gel or an agarose gel separates molecules on the basis of the molecule's size. A polymeric gel provides a porous passageway through which the molecules can travel. Polymeric gels permit the separation of molecules by molecular size because larger molecules will travel more slowly through the gel than smaller molecules.

In some embodiments, the sample containing a protein of interest is separated or resolved based on the charge of the components of the sample. In some embodiments, protein components of a sample are separated by charge and the method is a method of detecting and/or discriminating between charge variants of a monoclonal antibody of interest. In some embodiments, fragments of a sample are separated by charge and the method is a method of detecting and/or discriminating between charge variants of an antibody of interest of interest.

In some embodiments, an internal standard can be a purified form of the antibody of interest itself or fragment thereof, which is generally made distinguishable from the antibody of interest in some way. Methods of obtaining a purified form of the antibody of interest itself or fragment thereof can include, but are not limited to, purification from nature, purification from organisms grown in the laboratory (e.g., via chemical synthesis), and/or the like. The distinguishing characteristic of an internal standard can be any suitable change that can include, but is not limited to, dye labeling, radiolabeling, or modifying the mobility of the standard during the electrophoretic separation so that it is separated from the antibody of interest. For example, a standard can contain a modification of the antibody of interest itself or fragment thereof that changes the charge, mass, and/or length (e.g., via deletion, fusion, and/or chemical modification) of the standard relative to the antibody of interest itself or fragment thereof. Thus, the antibody of interest itself or fragment thereof and the internal standard can each be labeled with fluorescent dyes that are each detectable at discrete emission wavelengths, thereby allowing the protein of interest and the standard to be independently detectable. In some instances, an internal standard is different from the antibody of interest itself or fragment thereof but behaves in a way similar to or the same as the antibody of interest itself or fragment thereof, enabling relevant comparative measurements.

As will be appreciated by those of skill in the art, virtually any method of loading the sample in the capillary may be performed. For example, the sample can be loaded into one end of the capillary. In some embodiments, the sample is loaded into one end of the capillary by hydrodynamic flow. For example, in embodiments wherein the fluid path is a capillary, the sample can be loaded into one end of the capillary by hydrodynamic flow, such that the capillary is used as a micropipette. In some embodiments, the sample can be loaded into the capillary by electrophoresis, for example, when the capillary is gel filled and therefore more resistant to hydrodynamic flow.

The capillary can include any structure that allows liquid or dissolved molecules to flow. Thus, the capillary can include any structure known in the art, so long as it is compatible with the methods. In some embodiments, the capillary is a bore or channel through which a liquid or dissolved molecule can flow. In some embodiments, the capillary is a passage in a permeable material in which liquids or dissolved molecules can flow.

The capillary includes any material that allows the detection of the protein of interest within the capillary. The capillary includes any convenient material, such as glass, plastic, silicon, fused silica, gel, or the like. In some embodiments, the method employs a plurality of capillaries. A plurality of capillaries enables multiple samples to be analyzed simultaneously.

The capillary can vary as to dimensions, width, depth and cross-section, as well as shape, for example being rounded, trapezoidal, rectangular, etc. The capillary can be straight, rounded, serpentine, or the like. As described below, the length of the fluid path depends in part on factors such as sample size and the extent of sample separation required to resolve the protein of interest.

In some embodiments, the capillary includes a tube with a bore. In some embodiments, the method employs a plurality of capillaries. Suitable sizes include, but are not limited to, capillaries having internal diameters of about 10 to about 1000 μm, although more typically capillaries having internal diameters of about 25 to about 400 μm can be utilized. Smaller diameter capillaries use relatively low sample loads while the use of relatively large bore capillaries allows relatively high sample loads and can result in improved signal detection.

The capillaries can have varying lengths. Suitable lengths include, but are not limited to, capillaries of about 1 to 20 cm in length, although somewhat shorter and longer capillaries can be used. In some embodiments, the capillary is about 1, 2, 3, 4, 5, or 6 cm in length. Longer capillaries typically result in better separations and improved resolution of complex mixtures. Longer capillaries can be of particular use in resolving low abundance proteins of interest.

Generally, the capillaries are composed of fused silica, although plastic capillaries and PYREX (i.e., amorphous glass) can be utilized. As noted above, the capillaries do not need to have a round or tubular shape. Other shapes, so long as they are compatible with the methods described herein, may also be used.

In some embodiments, the capillary can be a channel. In some embodiments, the method employs a plurality of channels. In some embodiments, the capillary can be a channel in a microfluidic device. Microfluidics employ channels in a substrate to perform a wide variety of operations. The microfluidic devices can include one or a plurality of channels contoured into a surface of a substrate. The microfluidic device can be obtained from a solid inert substrate, and in some embodiments in the form of a chip. The dimensions of the microfluidic device are not critical, but in some embodiments the dimensions are on the order of about 100 μm to about 5 mm thick and approximately about 1 centimeter to about 20 centimeters on a side. Suitable sizes include, but are not limited to, channels having a depth of about 5 μm to about 200 μm, although more typically having a depth of about 20 μm to about 50 μm can be utilized. Smaller channels, such as micro or nanochannels can also be used, so long as they are compatible with the methods.

The antibody fragments may be obtained from an antibody of interest, such as a monoclonal antibody. The antibody fragments may be prepared by reduction, enzymatic digestion, denaturation, fragmentation, chemical cleavage or a combination thereof. The methods disclosed herein are applicable to any antibody isotype, such as IgG1, IgG2, IgG3, IgG4, or mixed isotype.

Reduction is to reduce disulfide bonds into two thiols in a 3-dimensional protein, such as a monoclonal antibody. Reduction can be performed by heat-denaturing, adding a surfactant, or adding a denaturing agent, e.g., guanidine HCl (6M), in the presence of a reducing agent, e.g. TCEP-HCl. Enzymatic degradation is a digestion of the protein with a protease, e.g., trypsin or Achromobacter protease I (Lys-C). In addition, the glycoprotein can be denatured by heat or chemicals, or a combination thereof. Fragmentation involves cleaving protein portions of a single or multi-subunit protein, such as a monoclonal antibody, with physical, biological or chemical methods. For example, an immunoglobulin degrading enzyme from S. pyogenes (IdeS) is commonly used for antibody subunit fragmentation.

In various embodiments, an antibody in a sample can be treated and prepared by reduction, enzymatic degradation, denaturation or fragmentation prior to contacting with the hydrophilic enrichment substrate. The methods provide a novel chromatographic method to characterize the post-translational modification of antibodies, e.g., monoclonal antibody (mAb) therapeutics, by means of fragments. In certain embodiments, the samples at any intervening step may be concentrated, desalted or the like.

In some embodiments, the methods further comprise detecting the post-translationally modified antibody fragments, for example using the UV signal from the peptide portion of the post-translationally modified antibody fragments. This may be done for fractions of a sample and allows the selection of specific fractions for further analysis, for example mass spec (MS) analysis. Thus, in further embodiments, the detection step comprises mass spectrometry or liquid chromatography-mass spectrometry (LC-MS). In applications of mass spectrometry for the analysis of biomolecules, the molecules are transferred from the liquid or solid phases to gas phase and to vacuum phase. Since many biomolecules are both large and fragile (proteins being a prime example), two of the most effective methods for their transfer to the vacuum phase are matrix-assisted laser desorption ionization (MALDI) or electrospray ionization (ESI). Aspects of the use of these methods, and sample preparation requirements, are known to those of ordinary skill in the art. In general, ESI is more sensitive, while MALDI is faster. Significantly, some peptides ionize better in MALDI mode than ESI, and vice versa (Genome Technology, June 220, p 52). The extraction channel methods and devices of the instant invention are particularly suited to preparing samples for MS analysis, especially biomolecule samples such as post-translationally modified antibody fragments. An important advantage of the invention is that it allows for the preparation of an enriched sample that can be directly analyzed, without the need for intervening process steps, e.g., concentration or desalting.

ESI is performed by mixing the sample with volatile acid and organic solvent and infusing it through a conductive needle charged with high voltage. The charged droplets that are sprayed (or ejected) from the needle end are directed into the mass spectrometer, and are dried up by heat and vacuum as they fly in. After the drops dry, the remaining charged molecules are directed by electromagnetic lenses into the mass detector and mass analyzed. In one embodiment, the eluted sample is deposited directly from the capillary into an electrospray nozzle, e.g., the capillary functions as the sample loader. In another embodiment, the capillary itself functions as both the extraction device and the electrospray nozzle.

For MALDI, the analyte molecules (e.g., proteins) are deposited on metal targets and co-crystallized with an organic matrix. The samples are dried and inserted into the mass spectrometer, and typically analyzed via time-of-flight (TOF) detection. In one embodiment, the eluted sample is deposited directly from the capillary onto the metal target, e.g., the capillary itself functions as the sample loader. In one embodiment, the extracted analyte is deposited on a MALDI target, a MALDI ionization matrix is added, and the sample is ionized and analyzed, e.g., by TOF detection.

In some embodiments, other ionization modes are used e.g. ESI-MS, turbospray ionization mass spectrometry, nanospray ionization mass spectrometry, thermospray ionization mass spectrometry, sonic spray ionization mass spectrometry, SELDI-MS and MALDI-MS. In general, an advantage of these methods is that they allow for the “just-in-time” purification of sample and direct introduction into the ionizing environment. It is important to note that the various ionization and detection modes introduce their own constraints on the nature of the desorption solution used, and it is important that the desorption solution be compatible with both. For example, the sample matrix in many applications must have low ionic strength, or reside within a particular pH range, etc. In ESI, salt in the sample can prevent detection by lowering the ionization or by clogging the nozzle. This problem is addressed by presenting the analyte in low salt and/or by the use of a volatile salt. In the case of MALDI, the analyte should be in a solvent compatible with spotting on the target and with the ionization matrix employed. In embodiments, the method further includes identifying the antibody fragments, for example the sequence of the antibody fragments. In embodiments, the method further includes identifying the post-translational modification present on the antibody fragments. In embodiments, the method further includes post-translational modification profiling of the antibody of interest. In embodiments, the method further includes post-translational modification mapping of post-translational modification hotspots by reduced peptide mapping LC-MS/MS analysis.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The following examples are provided to illustrate particular features of certain embodiments. However, the particular features described below should not be considered as limitations on the scope of the invention, but rather as examples from which equivalents will be recognized by those of ordinary skill in the art.

### EXAMPLES

The following examples are put forth so as to provide those of ordinary skill in the art with a complete disclosure and description of how to make and use the methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

Reagents and Materials. Deionized water was generated with a Milli-Q integral 10 water purification system with a MilliPak Express 20 filter (MilliporeSigma, Burlington, Mass.). NISTmAb (Reference material 8671, humanized IgG1K monoclonal antibody) was purchased from the National Institute of Standards and Technology (Gaithersburg, Md.). All other antibodies including mAb1-7 and bsAb1-3 were generated internally by Regeneron Pharmaceuticals, Inc. (Tarrytown, N.Y.). Dimethyl sulfoxide (DMSO, HPLC grade) was purchased from Thermo Fisher Scientific (Rockford, Ill.). Urea (BioXtra) was purchased from MilliporeSigma. ProteinSimple methyl cellulose (MC) solution at 0.5% and 1%, Pharmalytes (broad range pH 3-10), and pI markers were purchased from VWR International (Radnor, Pa.). ZipChip HRN chips with a 22 cm separation channel and a native assay kit with Native Antibodies background electrolyte (BGE) were acquired from 908 Devices (Boston, Mass.).

Sample preparation. To remove N-glycans from each heavy chain CH2 domain, NISTmAb sample was treated with PNGase F (1 IUB milliunit per 10 μg of protein) at 45° C. in Milli-Q water for 1 hour.

For bsAb impurity analysis, the concentration of each bsAb stock solution and two homospecific mAb impurities (referred to as HC/HC and HC*/HC*) were measured based on the UV absorbance at 280 nm and the corresponding extinction coefficient. A series of solutions were prepared through mixture of bsAb with percentages of homospecific mAb impurities at 0.2% and 1.0%.

Forced degradation study. NIST IgG1 mAb (5 mg/mL, pH 6.0) was incubated at 45° C. for 0, 1, 4, 8, 15 and 28 days.

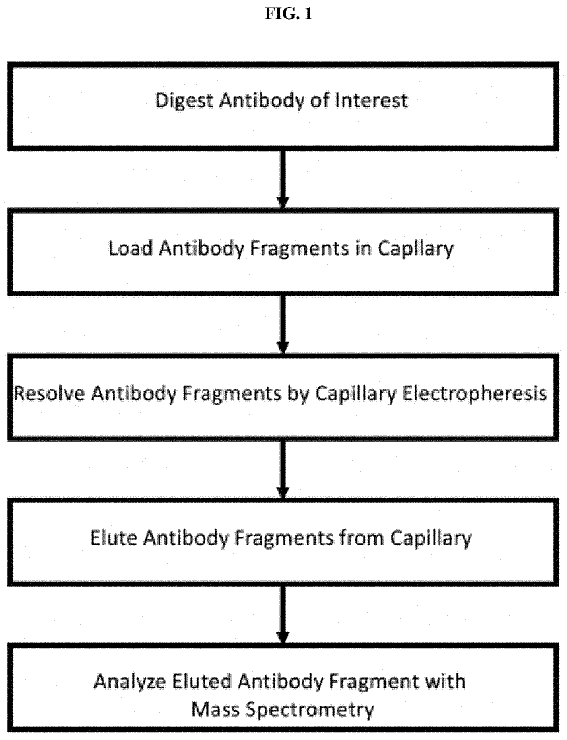

IdeS treatment. For subunit analysis, each NISTmAb sample was diluted to 2 mg/mL with Milli-Q water. Then 125 units of IdeS (Promega) was added to 100 μg of mAb at enzyme/antibody ratio of 1.25/1. The mixture was incubated at 37° C. with shaking at 600 rpm for 30 minutes to generate F(ab′)2 and Fc fragments. Control and stressed samples after each study/treatment were stored immediately at −20° C. A summary of the method is shown in FIG. 1.

Native microfluidic CE-MS (nCE-MS). The intact mass analysis of antibodies and charge variants was conducted with the ZipChip CE interface (908 Devices, Boston, Mass.) coupled to an Exactive Plus EMR Orbitrap mass spectrometer (Thermo Scientific, Bremen, Germany). Samples were loaded on the sample well of a microfluidic chip by a model 840 autosampler with a pressure assistance start time at 0.2 min and a replicate delay of 30 seconds. The injection volume was 1 nL. Charge variants were separated in the separation channel of the microfluidic chip by using Native Antibodies BGE, 3.8% DMSO, pH ˜5.5, under a field strength of 650 V/cm for 15 min. Each separated charge variant species was then ionized and subjected to MS analysis. MS data acquisition was performed through a full scan at a resolution of 17,500 in positive mode with a scan range of 1000-10000 m/z, in-source CID at 100 eV, AGC at 3e6, maximum injection time of 50 ms, and 3 microscans. The ESI parameters were set with spray voltage at 0, capillary temperature at 300° C., S-lens at 150, sheath gas at 2, auxiliary gas at 0, and trapping gas at 1.

Strong cation exchange chromatography. Antibody charge variants were separated on a MabPac SCX-10 column (4×250 mm) and an Agilent 1290 Infinity HPLC. The pH gradient was optimized according to the tested antibody's theoretical pI value. Mobile phase A (MPA) and mobile phase B (MPB) were CX-1 pH gradient buffer A and B with pH 5.6 and 10.2 (Thermo Fisher Scientific), respectively. Briefly, for IgG4 and bispecific IgG4 antibodies, the charge variants were separated with a pH gradient from 5% to 15% MPB in 30 min. For IgG1 antibodies, the pH gradient was adjusted from 45% to 70% MPB in 30 min. Absorbance was measured at a UV wavelength of 280 nm. The flow rate was set to 0.8 mL/min, and the column temperature was set to 25° C.

Imaged capillary isoelectric focusing. The final sample solution contained 0.5 mg/mL protein, 4% (v/v) pH 3-10 Pharmalytes, 2 M urea, and 0.35% (w/v) methylcellulose. Samples were prepared in triplicate in the presence or absence of pI markers. Each sample was hydrodynamically injected into a fluorocarbon-coated silica capillary (100 mm inner diameter, 50 mm observable window, ProteinSimple, San Jose, Calif.) at 2 bar for 70 seconds with a ProteinSimple iCE3 system. Samples were subsequently pre-focused for 1 minute at 1.5 kV, then subjected to a 7.0 minute focusing period at 3 kV with 100 mM sodium hydroxide as the catholyte and 80 mM phosphoric acid as the anolyte, both in 0.1% (w/v) methyl cellulose. Proteins were monitored during focusing according to the UV absorbance at 280 nm with a scanning charge-coupled device (CCD) camera. Electropherograms were generated with CCD detection of UV light absorption across the separation capillary.

Data analysis. Raw MS spectra were analyzed in Thermo Xcalibur 4.3.73.11 and deconvoluted with Intact Mass™ software version 3.11-1 (Protein Metrics Inc., Cupertino, Calif.) for charge variant analysis. Base peak chromatograms (BPCs) were processed with Microsoft Excel macros for the same mAb experiment. For data acquired with an Agilent HPLC system, the data were analyzed in OpenLAB CDS ChemStation Edition Rev. C.01.07 SR2.

### Example 1. Investigation of Sensitivity of nCE-MS for Therapeutic Antibodies

This disclosure sets forth the development of a high-resolution, high-sensitivity and high-throughput native capillary electrophoresis (CE)-mass spectrometry (MS) method for antibody charge heterogeneity analysis. A panel of fifteen antibodies, including IgG1, IgG4 and bispecific mAbs, were analyzed using the Zipchip nCE-MS method.

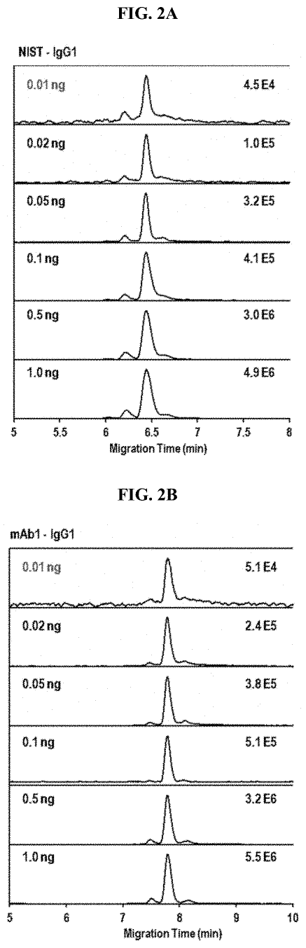

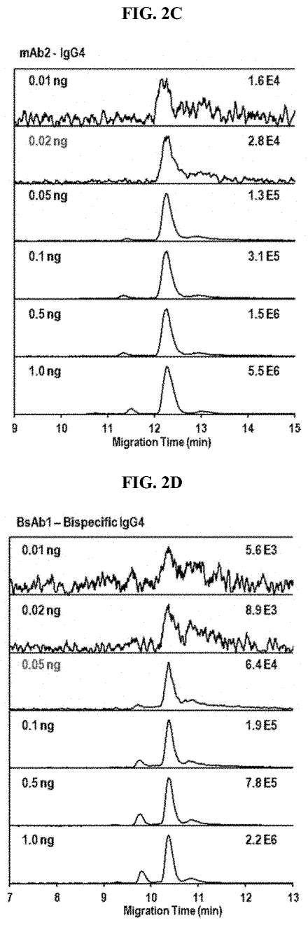

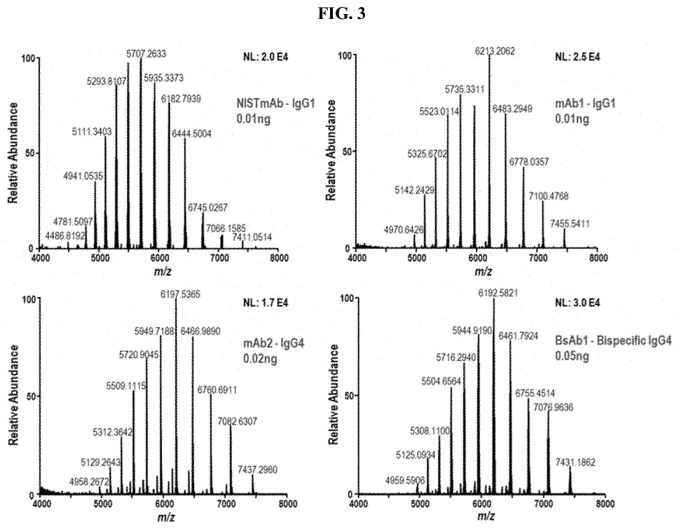

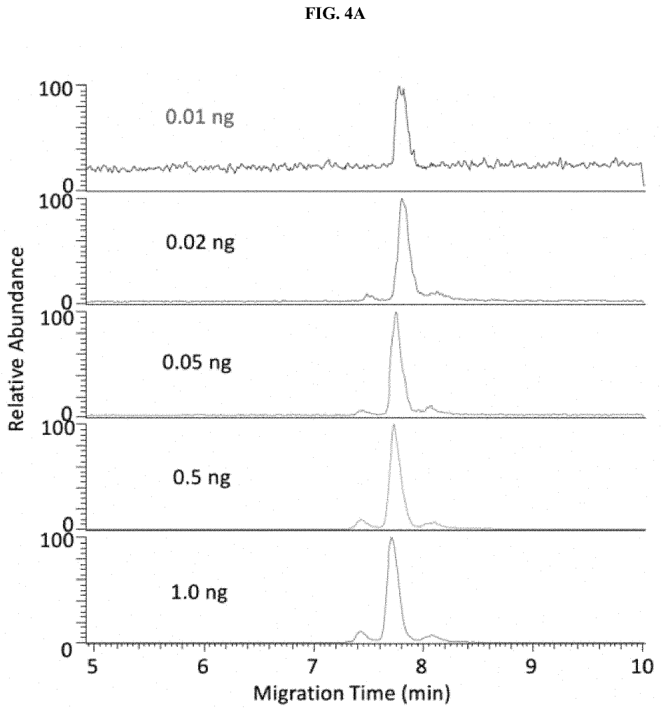

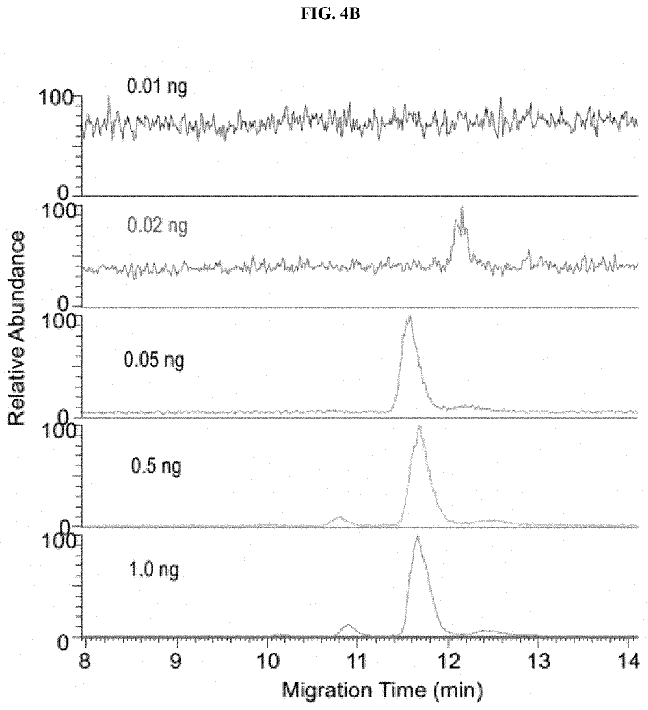

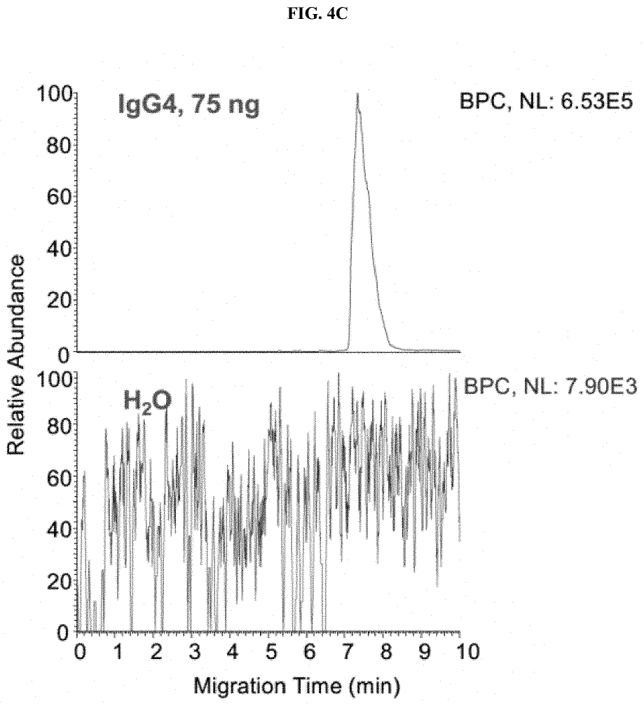

ZipChip nCE-MS combined with nanospray ESI provides ultra-high sensitivity in detecting low abundant charge variant species. The method sensitivity of nCE-MS in analyzing various types of IgG was evaluated by separate injection of different amounts of antibody from 0.01 ng to 1 ng on the chip. The antibody charge separation profile was extracted with BPCs with mass range from 4000 to 9000 m/z, as shown in FIG. 2. In the case of IgG1 antibodies for both NISTmAb and mAb1, a limit of detection (LOD) of 0.01 ng was achieved, as shown in FIG. 2A, FIG. 2B, and FIG. 4A. For IgG4, mAb2 and bsAb1, the MS signal was detected down to 0.02 ng and 0.05 ng, respectively, as shown in FIG. 2C, FIG. 2D, and FIG. 4B. At the LOD for all four mAbs including IgG1, IgG4, and bispecific IgG4, the mass spectra of the main species of each antibody showed a high signal-to-noise ratio, thus demonstrating the excellent sensitivity of the nCE-MS method, as shown in FIG. 3. Moreover, the charge variant profile was directly observed at the LOD for all four mAbs. No run-to-run injection carryover was observed for the nCE-MS method, as shown in FIG. 4C.

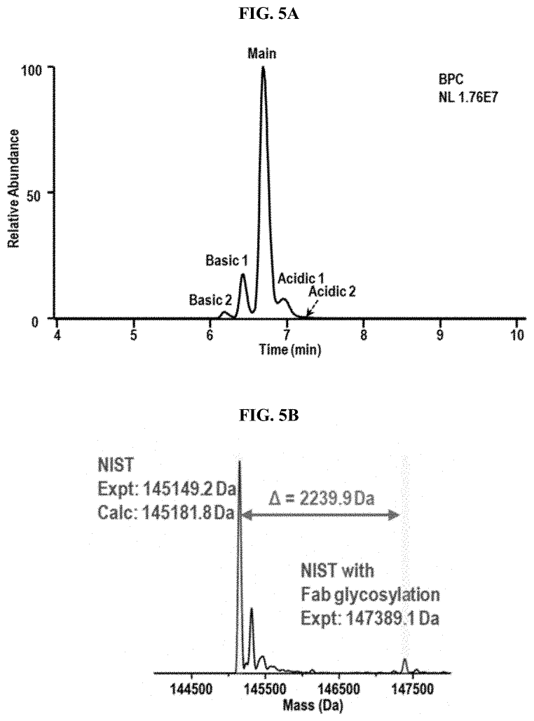

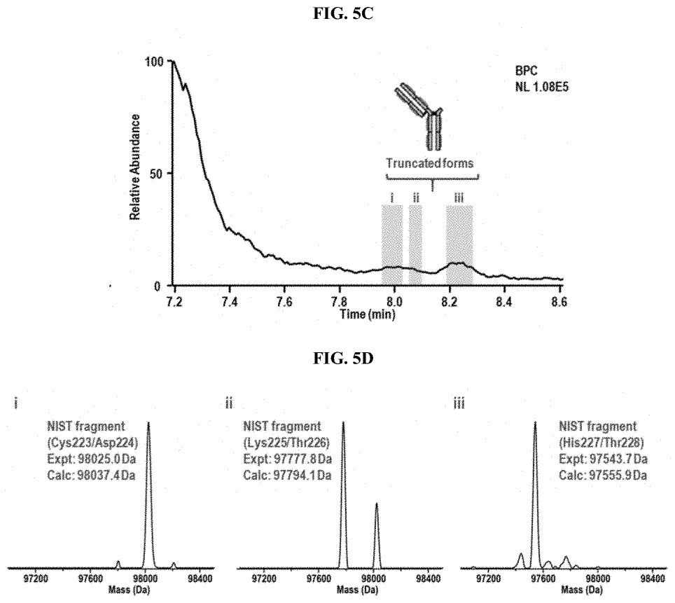

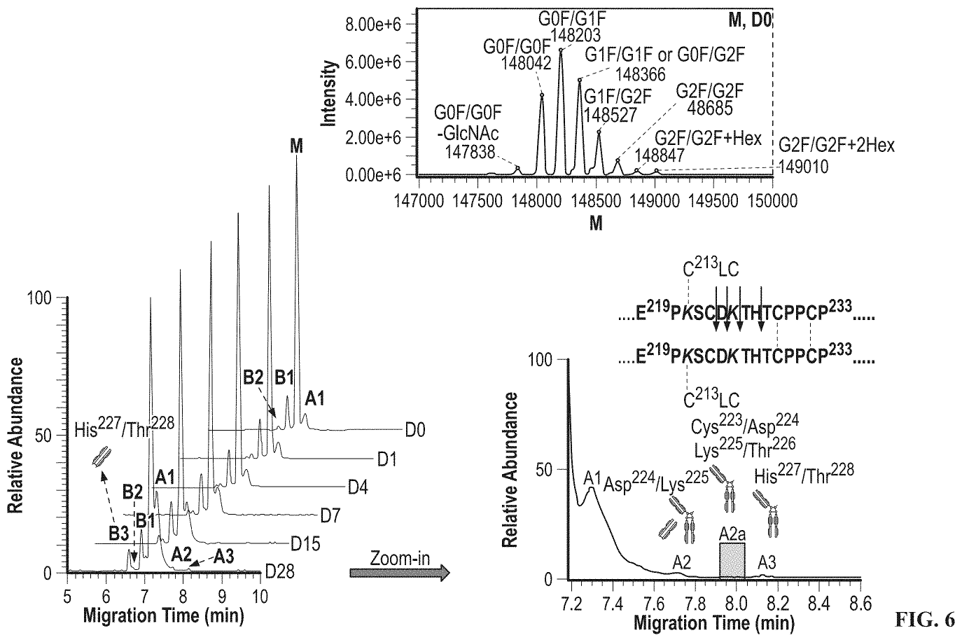

The high sensitivity of the nCE-MS method was also demonstrated by the observation of truncation species with low amounts of sample injection. A deglycosylation reaction step removed all N-glycans attached to the mAb, thus decreasing the proteoform complexity and increasing the sensitivity to detect sequence variations. The electropherogram of deglycosylated NISTmAb showed identical charge variant profile and identifications to those of intact NISTmAb, as shown in FIG. 2A and FIG. 5A. In the A2 acidic variant, Fab glycosylation was detected with a 2 ng injection, as shown in FIG. 5B (Yan et al., 2018). Additionally, three truncation species on the upper hinge region were detected including the cleavage at Cys223/Asp224, Lys225/Thr226, and His227/Thr228, as shown in FIGS. 5C and 5D, thus demonstrating the high sensitivity of the nCE-MS method (Yan et al., 2018).

In order to assess the sensitivity of the method for additional fragmented and truncated species, a forced degradation study and subunit digestion study was conducted using NISTmAb. The NISTmAb reference standard and its heat-stressed forms were analyzed following incubation at 45° C. for up to 28 days. Samples with different incubation times were cleaved by IdeS digestion to generate F(ab′)2 or Fc associated subunit species. The intact mass analysis of both control and stressed antibodies was conducted using a universal Zipchip nCE-MS method. The PTM hotspots were identified by reduced peptide mapping LC-MS/MS analysis to elucidate the elevated charge variants under stressed conditions. Intact mass data was processed by PMi-Intact software. The elevated charge variants were allocated to F(ab′)2 or Fc by the subunit charge variant analysis. Reduced peptide mapping data was processed by BioPharma Finder 3.0 and Skyline-daily 4.2 for PTMs identification and quantification, respectively.

Deconvoluted mass data indicated that 4 major Fab cleavages at the upper hinge region of NISTmAb were generated upon 28-day heat stress, in addition to significantly increased acidic variant (A1) compared to the control (FIG. 6 and Table 2).

TABLE 2
Summary of charge variant identification

| Charge | | Stressed NISTmAb |
|---|---|---|
| Variants | Control NISTmAb | (45° C., pH 6.0, 28 days) |
| Basic 1 (B1) | +1 C-terminal lysine | +1 C-terminal lysine |
| Basic 2 (B2) | +2 C-terminal lysine | +2 C-terminal lysine |
| Basic 3 (B3) | ND | Fab cleavage at His²²⁷/Thr²²⁸ |
| Acidic 1 (A1) | +Deamidation | +Deamidation |
| Acidic 2 (A2) | ND | Fab cleavage at Asp²²⁴/Lys²²⁵ |
| Acidic 2a | ND | Loss of Fab cleaved at Cys²²³/ |
| (A2a) | | Asp²²⁴ and Lys²²⁵/Thr²²⁶ |
| Acidic 3 (A3) | ND | Loss of Fab cleaved at His²²⁷/ |
| | | Thr²²⁸ |

Note:
ND—not detected

Acidic and basic charge variants were well separated from the main peak for the NIST antibody standard by Zipchip CE running at native conditions. The MS analysis identified two basic variants corresponding to the antibody with 1 and 2 unprocessed C-terminal lysines on the heavy chain, while an acidic variant was mainly caused by deamidation.

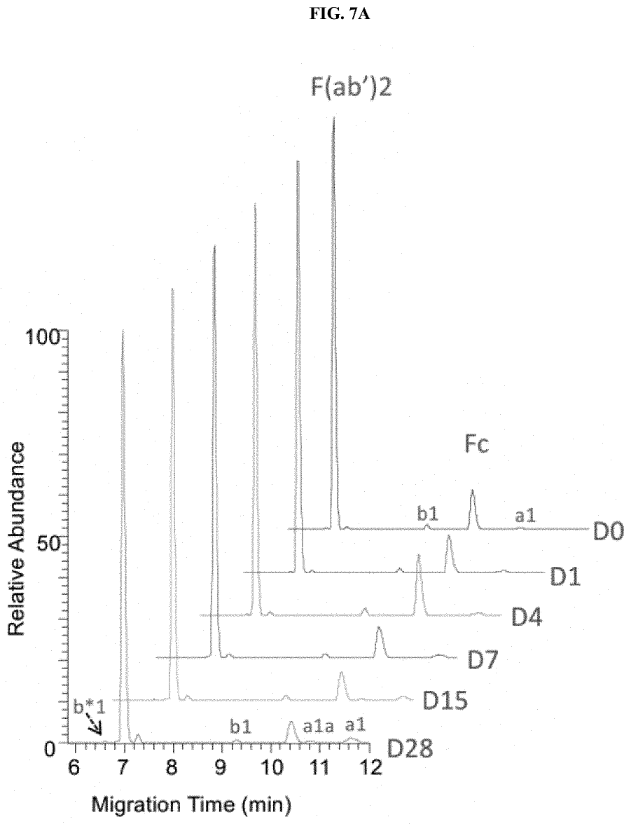

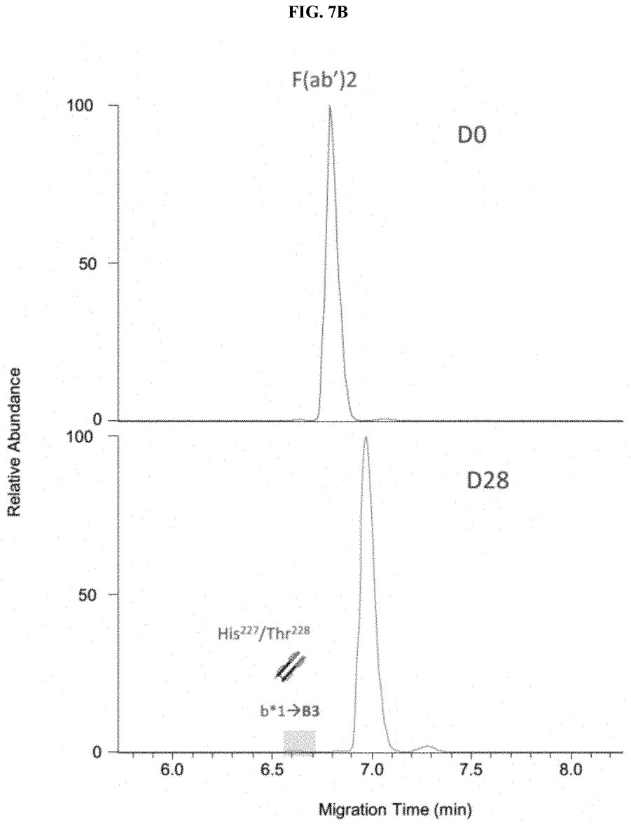

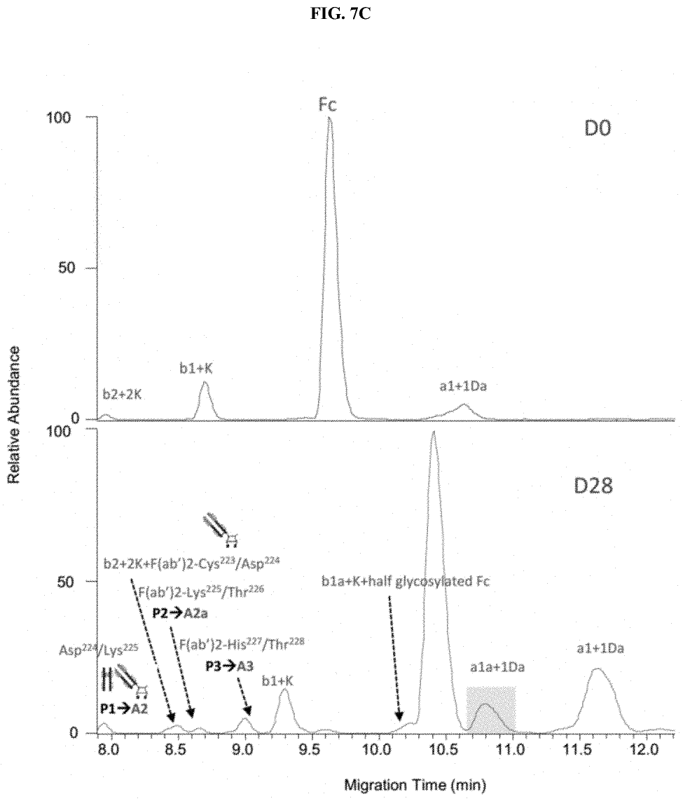

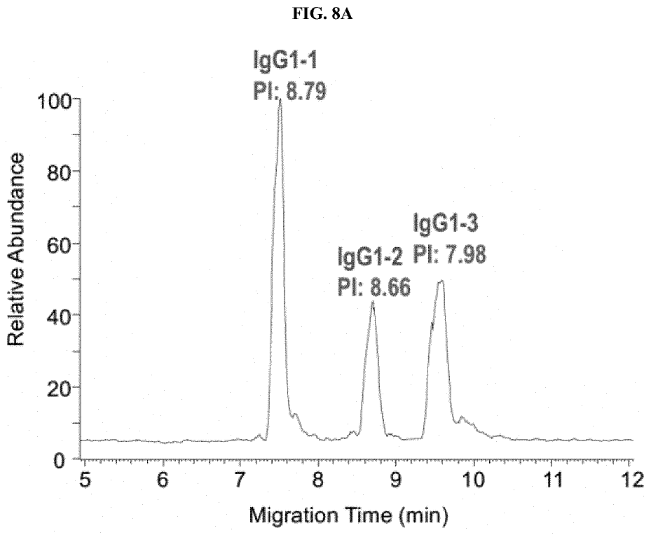

F(ab′)2 and Fc were well separated by the universal nCE-MS method. All minor peaks were identified, as shown in FIGS. 7A-7C. New charge variants resulting from 28-day incubation are indicated in blue shaded regions. Two basic variants with 1 and 2 unprocessed C-terminal lysines were located in the Fc region (FIG. 7C). The basic variant 3, resulting from Fab cleavage at His²²⁷/Thr²²⁸ in stressed sample (45° C., D28), was identified as F(ab′)2 basic variant (b*1) in FIG. 7B. All other Fab cleavage sites were found in the acid region of F(ab′)2 and in the same order as those were identified during intact antibody analysis. For Fc acidic region, a new acidic variant A1a caused by Asp isomerization showed up in D28 sample. Compared to main Fc, the +1 Da mass increase of both A1 and A1a indicated that the acidic variant might be caused by deamidation. This was confirmed by peptide mapping, as shown in Table 3.

TABLE 3
Summary of major PTMs showing differences under stressed conditions

| Antibody | | | D 0 | | | | | |
|---|---|---|---|---|---|---|---|---|
| Region | PTMs | Site | (Control) | D 1 | D 4 | D 7 | D 15 | D 28 |
| Fc | Deamidation | HC Asn387 | 1.61% | 1.64% | 1.71% | 1.91% | 2.15% | 2.64% |
| | Deamidation | HC Asn392 | 0.75% | 0.83% | 1.04% | 1.40% | 1.99% | 3.03% |
| | Oxidation | HC Met255 | 1.23% | 1.56% | 1.66% | 1.88% | 2.05% | 3.00% |
| | Isomerization | HC Asp283 | 0.65% | 0.70% | 0.87% | 1.29% | 1.70% | 2.81% |
| F(ab′)2 | Isomerization | LC Asp166 | 2.65% | 3.28% | 3.58% | 4.64% | 5.59% | 7.62% |

### Example 2. Separation of Antibody Mixtures for High-Throughput Intact Mass Analysis

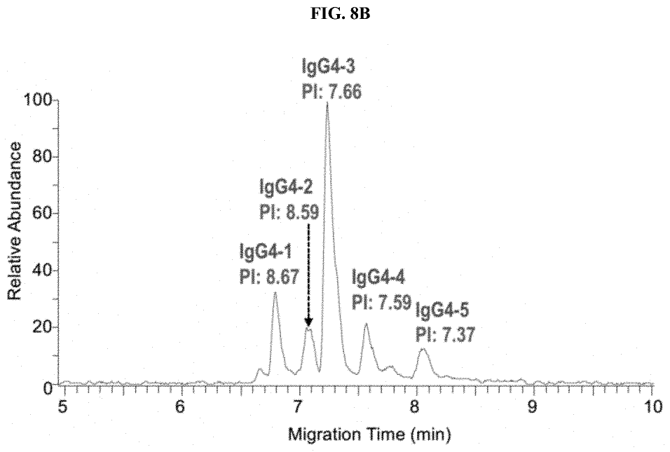

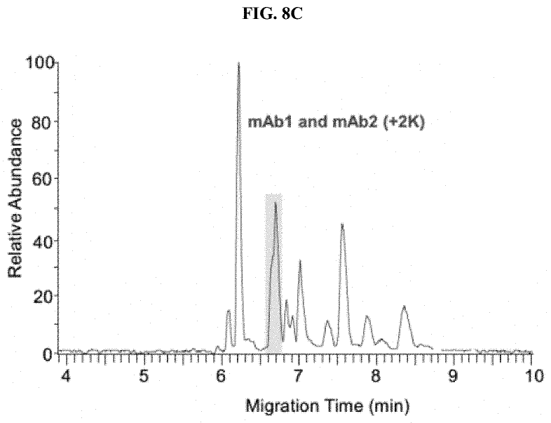

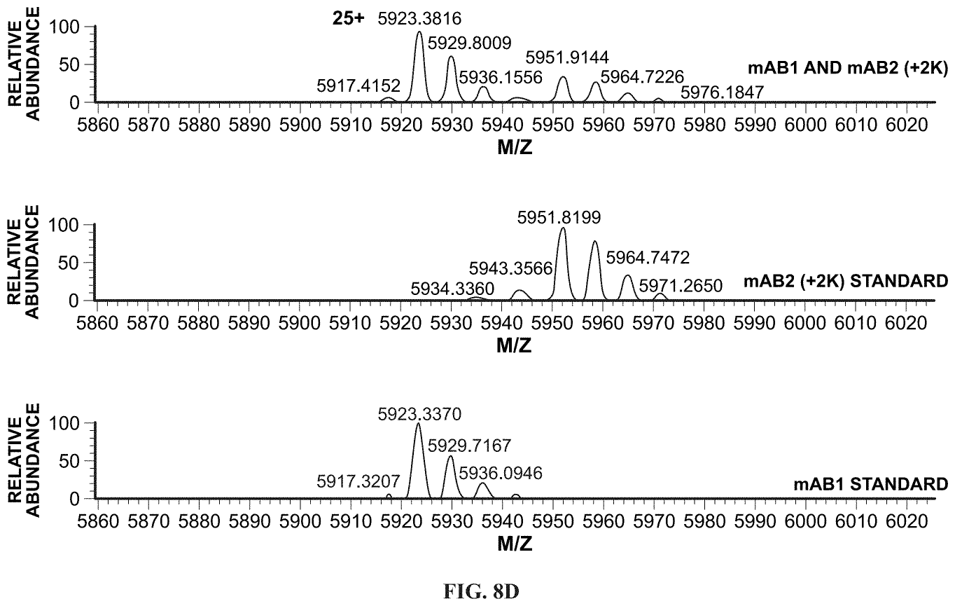

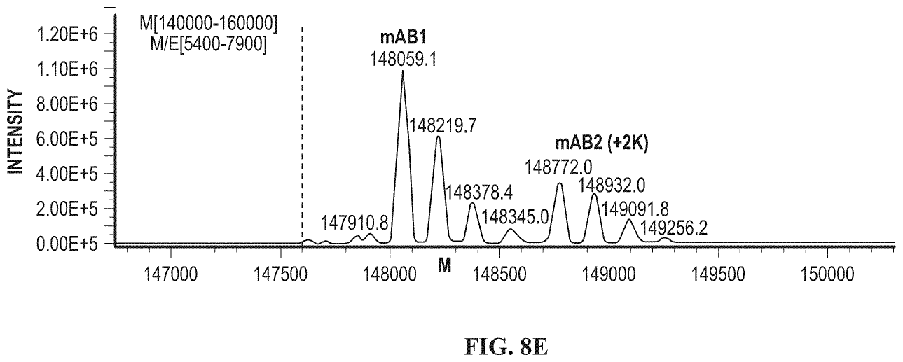

Three IgG1 mAbs in mixture 1 (FIG. 8A) and five IgG4 bispecific mAbs in mixture 2 (FIG. 8B) were well separated by the ZipChip nCE-MS method of the present invention, which can be applied for intact mass analysis of co-formulated drugs. Even if two antibodies co-migrated together (blue shaded area in FIG. 8C), those can be identified individually from native MS data, as shown in FIG. 8D and FIG. 8E. FIG. 8E shows the convoluted spectra. Besides high-resolution charge variant analysis of a single antibody, the ZipChip nCE-MS method can also be used as a high-throughput and high-sensitivity approach for intact mass analysis of antibody mixture and ADCs.

### Example 3. System Robustness

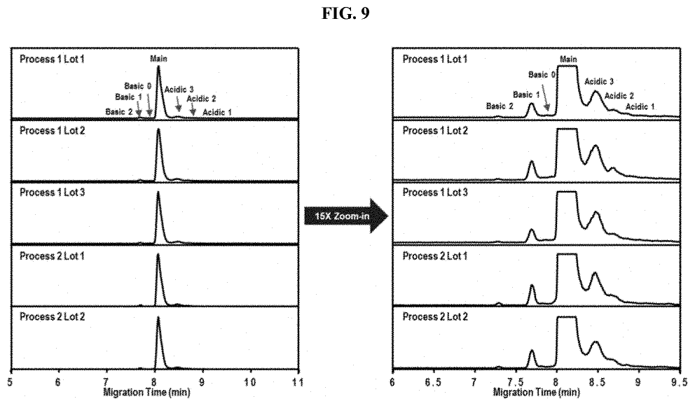

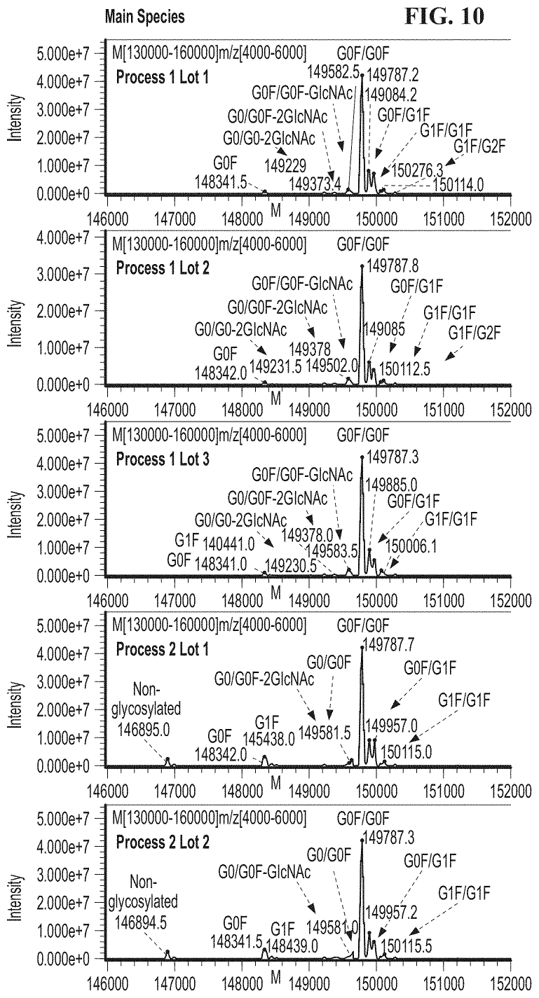

System robustness is important to assess when method is applied in various analytical tasks in industrial settings. This property is particularly crucial for comparability studies such as cell line selection and process optimization. As shown in FIG. 9 for an IgG4 mAb3, three lots from process 1 and two lots from process 2 were subjected to nCE-MS analysis. The charge variant profiles from the nCE-MS analysis of the five samples were highly comparable, showing three basic variants and three acidic variants. Moreover, MS analysis allowed for PTM characterization and the identification of processes related PTM differences, as shown in Table 4.

TABLE 4
Mass table for charge variant characterization of mAb3

| Charge Variant | |
|---|---|
| Peaks | Proposed Charge Variant Identity |
| Basic 2 | 2 × C-terminal Lys |
| | 2 × Gly loss with amidation |
| Basic 1 | 1 × C-terminal Lys |
| | 1 × Gly loss with amidation |
| Basic 0 | 2 × N-terminal pyroglutamate |
| Main | mAb |
| Acidic 3 | Deamidation |
| | Glycation |
| Acidic 2 | Deamidation |
| | Glycation |
| | 1 × NeuAc |
| Acidic 1 | Glycation |

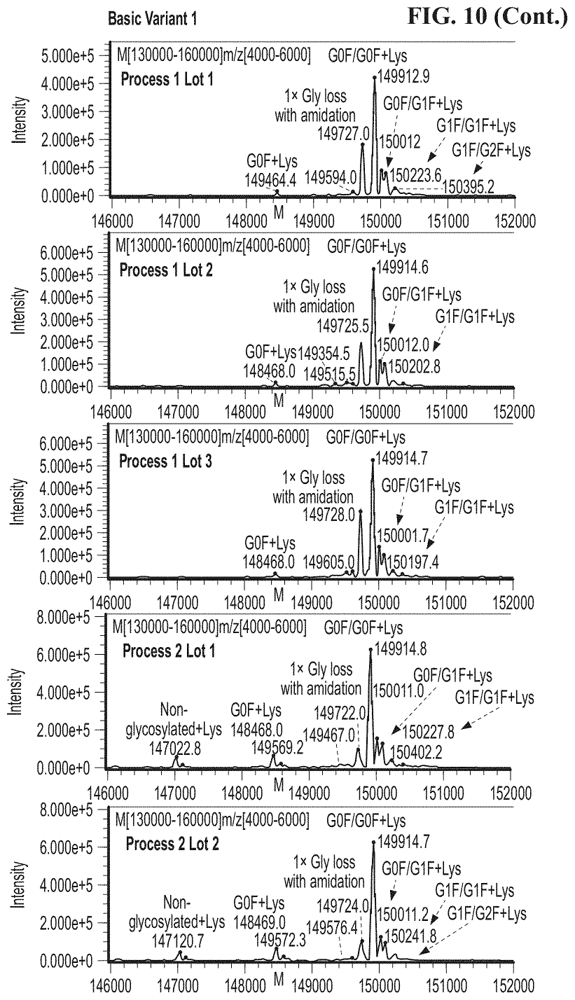

In the main species, more non-glycosylated species were observed in samples from process 2 than process 1, as shown in FIG. 10A. Additionally, higher levels of glycine loss with amidation were observed in samples from process 1 than process 2, as shown in basic variant 1, as shown in FIG. 10B. The highly comparable charge variant profile, in addition to the consistent results among samples from the same process, demonstrated the system robustness of nCE-MS analysis, which is suitable for comparability studies.

### Example 4. Comparison of Antibody Charge Variant Separation Among nCE-MS, SCX-UV, and iCIEF

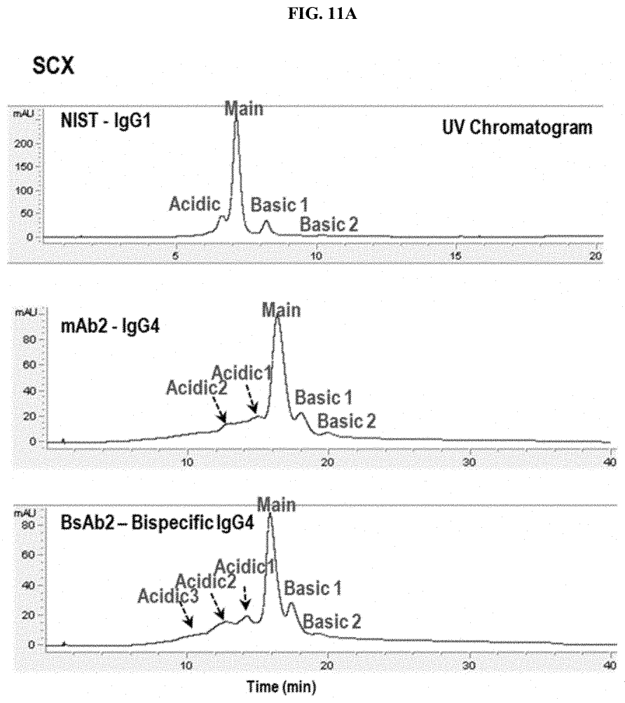

nCE-MS is a new method for charge variant analysis, in contrast to SCX and iCIEF, which are commonly used as characterization and release assays. In this study, the nCE-MS method was first compared to SCX with UV detection. Three types of antibodies including IgG1 NISTmAb, IgG4 mAb2, and bispecific IgG4 bsAb2 were used for this comparison. The charge variant separation profiles of these three types of antibodies are shown in FIG. 11 between the SCX-UV method using a pH gradient and nCE-MS. Comparable high-resolution charge variant separations of all types of IgG were obtained between platforms, including the same number of basic and acidic variants. Compared with SCX-UV, which usually requires injection of more than 10 μg, the nCE-MS method enabled high resolution charge variant separation with only a 1 ng injection. More importantly, each charge variant was directly identified by online MS analysis. In comparison, the SCX-UV method generally uses MS-incompatible salts, and often requires either fraction collection or two-dimensional liquid chromatography coupled with MS to elucidate the PTMs in the charge variants (Alvarez et al., 2011, Anal. Biochem., 419(1):17-25; Jaag et al., 2021, J. Chromatogr. A, 1636:461786).

Recently, SCX coupled to native MS (SCX-nMS) has been used for charge variant analysis of mAbs (Yan et al., 2018; Ma et al.; Haberger et al., 2021, J. Am. Soc. Mass Spectrom., 32(8):2062-2071). Similar charge variant profiles have been obtained between SCX-nMS and nCE-MS analysis (Fussl et al., 2020, Anal. Chem., 92(7):5421-5438). However, sample amounts of approximately 50 μg are often required for SCX-nMS with post-column flow splitting to enhance sensitivity, and to provide the same detection of truncated species and Fab glycosylation species shown in FIG. 5 (Yan et al., 2018; Ma et al.). In comparison, only a 2 ng injection (with 10 μg transferred to the sample well) was required with the integrated nCE-MS method. Additionally, the unused portion of the protein sample could be recovered from the ZipChip interface after the nCE-MS experiment and subjected to other assays, thus providing experimental flexibility, particularly in the drug discovery stage and other situations in which protein amounts are limited.

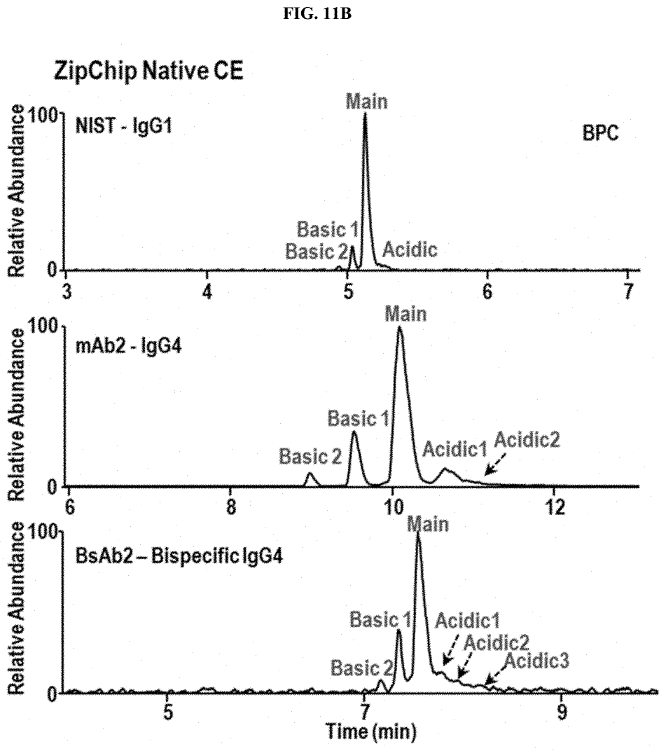

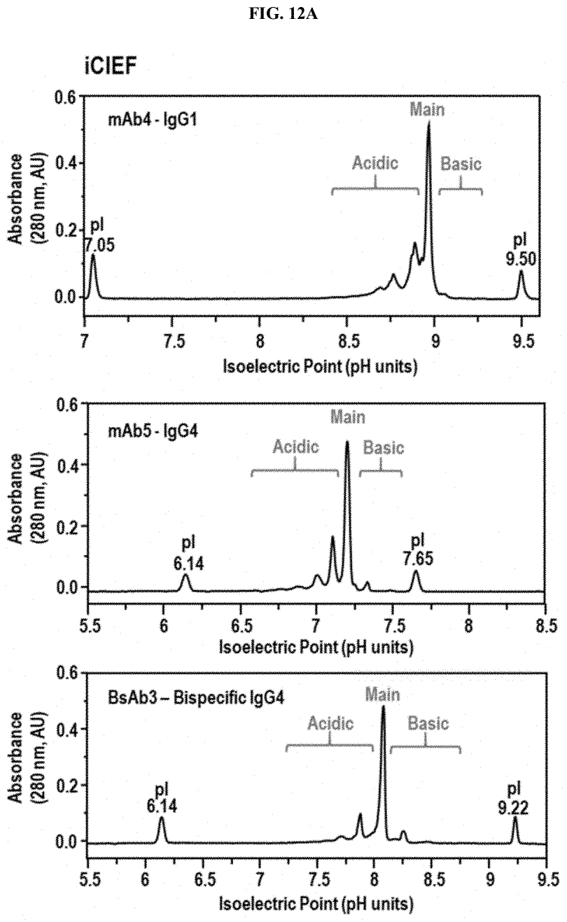

Charge variant separation between iCIEF and nCE-MS was also compared. Three types of antibodies including IgG1 mAb4, IgG4 mAb5, and bispecific IgG4 bsAb3 were used to compare these two methods. As shown in FIG. 12, the charge variants between the iCIEF and nCE-MS analysis showed several differences. In the case of IgG1 mAb4, the number of acidic variants between these two methods differed and the nCE-MS methods showed fewer acidic species than the iCIEF method, as shown in FIG. 12A. In contrast, the basic variant was more clearly observed in the nCE-MS method than the iCIEF method. In the case of both IgG4 mAb5 and bispecific IgG4 bsAb3, comparable electropherograms were observed between these two methods, as shown in FIGS. 12B and 12C. A recent study has suggested that electropherograms between iCIEF and nCE-MS analysis are comparable (Cao et al.). In the examples provided in this study, differences in electropherograms were observed, such as for IgG1 mAb4. The nCE-MS method also showed relatively lower resolution separation in the acidic region than iCIEF analysis, possibly because of tailing from the main species and potential diffusion caused by peak broadening during the long migration time in the separation channel. The differences are likely due to the different nature for separation between these two methods, where iCIEF separates analytes according to intrinsic pI whereas nCE-MS method is based on capillary zone electrophoresis that takes into account both the solvated charge and size of the analytes. Recently, direct coupling of iCIEF and MS has been used to accelerate peak identification for iCIEF analysis; however, the method remains under development, and wide adoption of this technique has yet to occur (Mack et al.; He et al.; Dai et al., 2018, Anal. Chem., 90(3):2246-2254). According to the observations in this study, nCE-MS is a highly sensitive, high-resolution approach for the characterization of antibody charge variants, and the similarity in electropherograms between iCIEF and nCE-MS may aid in the identification of antibody charge variants for iCIEF assays.

Despite the advantages of nCE-MS analysis, iCIEF and SCX assays are performed with UV detection, which can directly enable quantification of charge variants, whereas nCE-MS relies on BPCs from MS and provides the relative quantification. Additionally, the relative quantification is also impacted by the differential ionization efficiency among acidic, main, and basic species. The SCX-nMS method with flow splitting to a UV detector can be used to quantitatively measure the percentages of charge variants (Yan et al., 2018).

### Example 5. Bispecific Antibody Impurity Analysis

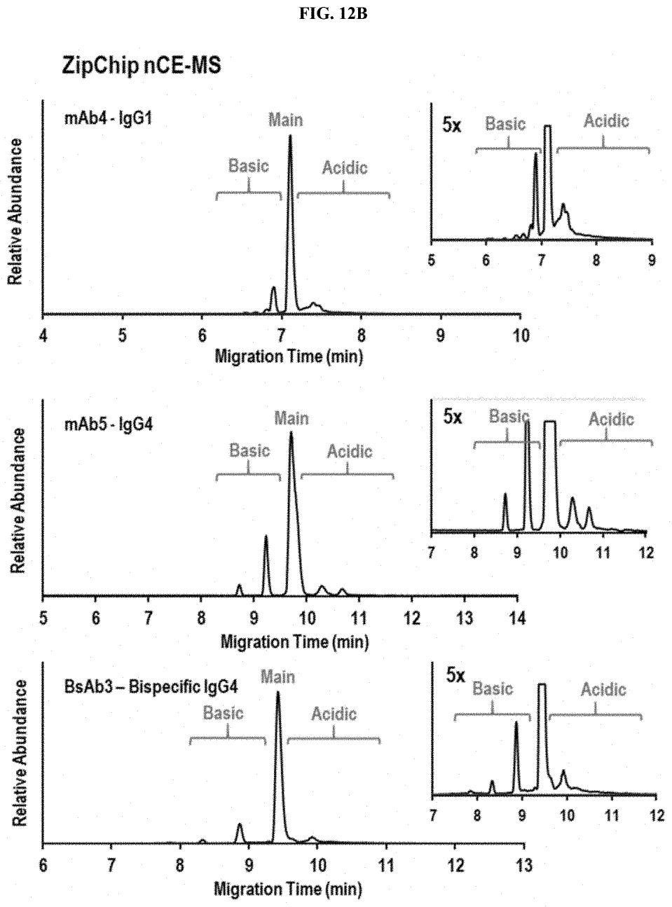

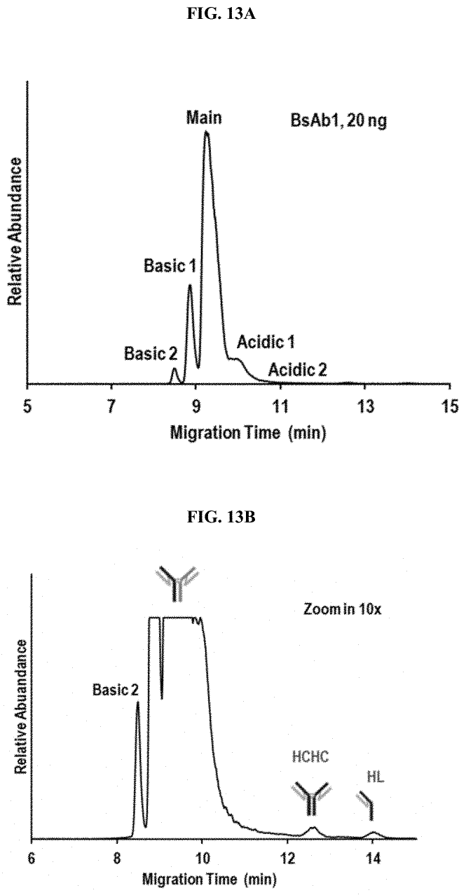

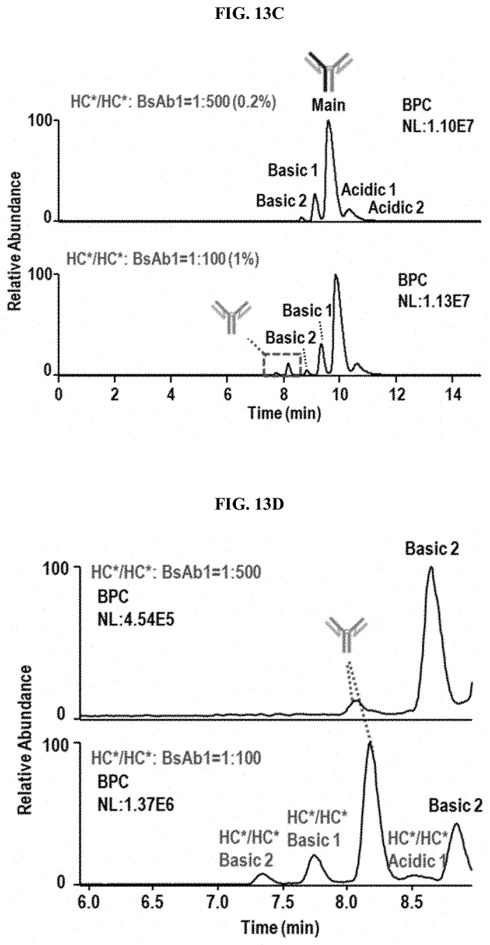

Impurities in bsAb therapeutics are a subject of interest for the pharmaceutical industry (Yan et al., 2019, Anal. Chem., 91(17):11417-11424). In this study, the bsAb of interest comprised two identical light chains and two different heavy chains, HC and HC* (Smith et al., 2015, Sci. Rep., 5(1):17943; Tustian et al., 2016, MAbs, 8(4):828-838). Although homospecific mAb impurities, which are composed of two identical light chains and two identical heavy chains (either HC or HC*), are removed during purification in the manufacturing process, some residual homospecific mAb impurities (referred to as HC/HC and HC*/HC*) remain in the desired bsAb product (Li, 2019, Protein Expr. Purif., 155(112-119). Quantification of these homospecific mAb species is necessary to ensure product safety and efficacy. The nCE-MS method was used for the analysis of the bispecific IgG4 bsAb1, as shown in FIG. 13A. For this bispecific antibody, common charge variants were detected, including antibody with one or two unprocessed C-terminal lysine residues in the basic variants, and deamidation, glycation, as well as one or two N-acetylneuraminic acid residues in the acidic variants, as shown in Table 5.

TABLE 5
Mass table for charge variant characterization of bsAb1

| Charge Variant | |
|---|---|
| Peaks | Proposed Charge Variant Identity |
| Basic 2 | 2 × C-terminal Lys |
| Basic 1 | 1 × C-terminal Lys |
| Main | mAb |
| Acidic 1 | Deamidation |
| | Glycation |
| | 1 × NeuAc |
| Acidic 2 | Deamidation |
| | Glycation |
| | 1 × NeuAc |
| | 2 × NeuAc |
| Others | HC/HC homospecific mAb impurities |
| | HL half antibody |

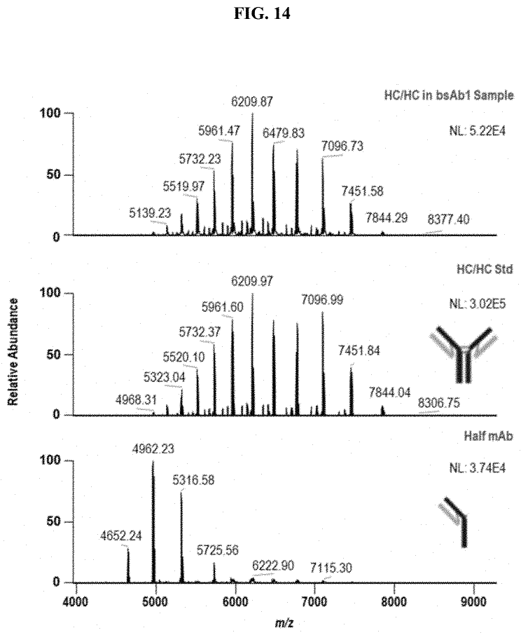

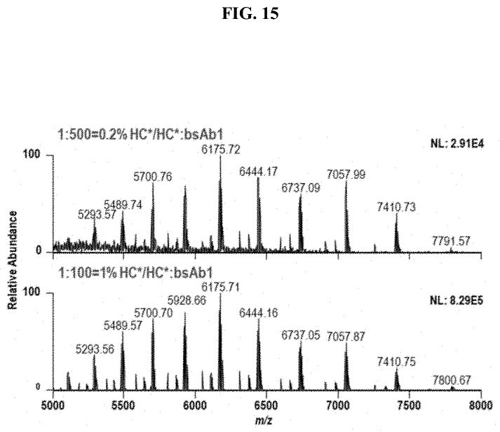

Interestingly, in the zoom-in view, low abundant HC/HC and half antibodies were observed after 20 ng injection into the nCE-MS, thus demonstrating the high sensitivity of the nCE-MS method, as shown in FIG. 13B and FIG. 14. However, HC*/HC* was not detected in the sample. To understand the relative abundance of HC*/HC* impurity in the bsAb1, bsAb1 sample was spiked with HC*/HC* impurity at a ratio of 1:500 (0.2%) and 1:100 (1%) and then analyzed using nCE-MS. Because the pI values of the HC and HC* are different, HC/HC and HC*/HC* impurities and bsAb should be separated in the nCE-MS method. In this spiking experiment, the HC*/HC* impurity was detected at a level of 0.2% in spiked bsAb1 samples, as shown in FIG. 13C and FIG. 15. The high sensitivity of the nCE-MS method was also demonstrated by the observation of separate peaks for charge variants of HC*/HC* impurity at a level of 1%, as shown in FIG. 13D. With this method, the HC*/HC* impurity was determined to be below 0.2%, if any, in the bsAb1 sample.

### Example 6. Charge Heterogeneity Analysis of New Formats of Biotherapeutics

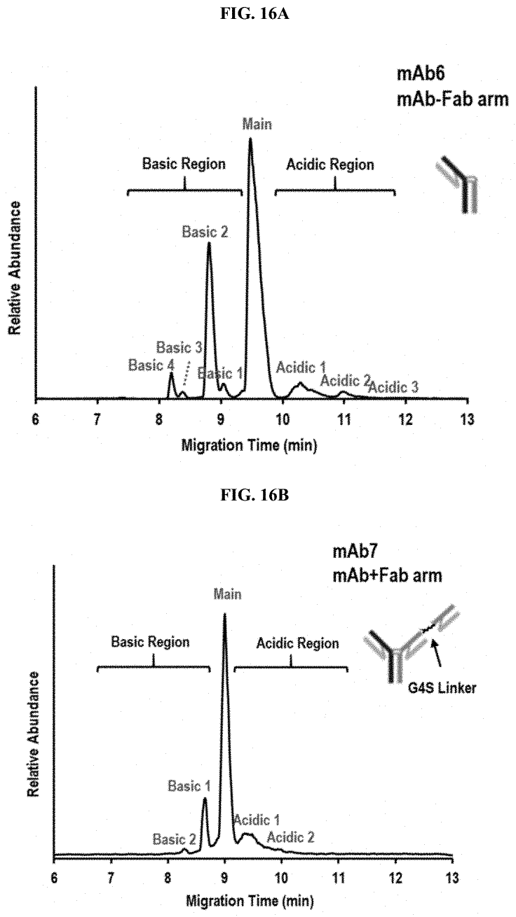

In addition to traditional formats of mAbs, including IgG1, IgG4, and bispecific antibodies described in the preceding sections, nCE-MS can analyze the charge variants of new formats of antibody derivatives. For example, mAb6 is a therapeutic antibody in “N−1” format where one Fab arm is removed, and it has Gln in the N-terminus of the HC. During mAb manufacturing, Gln would be converted into pyroglutamate. However, a small percentage of Gln is often found to be unconverted, thus resulting in a basic charge variant relative to the main species, which is a mAb with complete conversion of Gln to pyroglutamate (Liu et al., 2019, J. Pharm. Sci., 108(10):3194-3200). nCE-MS analysis revealed eight species, including four basic variants and three acidic variants, as shown in FIG. 16A and Table 6.

TABLE 6
Mass table for charge variant characterization
of mAb6 in “N-1” format

| Charge Variant | |
|---|---|
| Peaks | Proposed Charge Variant Identity |
| Basic 4 | 2 × C-terminal Lys |
| Basic 3 | 1 × C-terminal Lys and 1 × unconverted Gln |
| Basic 2 | 1 × C-terminal Lys |
| Basic 1 | 1 × unconverted Gln |
| Main | mAb |
| Acidic 1 | Deamidation |
| | Glycation |
| | 1 × NeuAc |
| Acidic 2 | 1 × NeuAc |
| | 2 × NeuAc |
| Acidic 3 | 2 × NeuAc |
| | 3 × NeuAc |

In the basic variants, PTMs including one or two unprocessed C-terminal Lys residues, one unconverted Gln, and a combination of one unprocessed C-terminal Lys residue and one unconverted Gln were observed. Three acidic variants included PTMs such as deamidation, glycation, and one to three N-acetylneuraminic acid residues.

For mAb7, an additional Fab arm was connected to the normal mAb with G4S inkers, and it is referred as “N+1” format. nCE-MS analysis revealed five species, including two basic variants and two acidic variants, as shown in FIG. 16B and Table 7.

TABLE 7
Mass table for charge variant characterization
of mAb7 in “N + 1” format

| Charge Variant | |
|---|---|
| Peaks | Proposed Charge Variant Identity |
| Basic 2 | 2 × C-terminal Lys |
| Basic 1 | 1 × C-terminal Lys |
| Basic 0 | 1 × N-terminal pyroglutamate |
| Main | mAb |
| Acidic 1 | Glycation |
| | O-glycan (Xyl + Gal + Gal + GlcA) |
| Acidic 2 | Glycation |

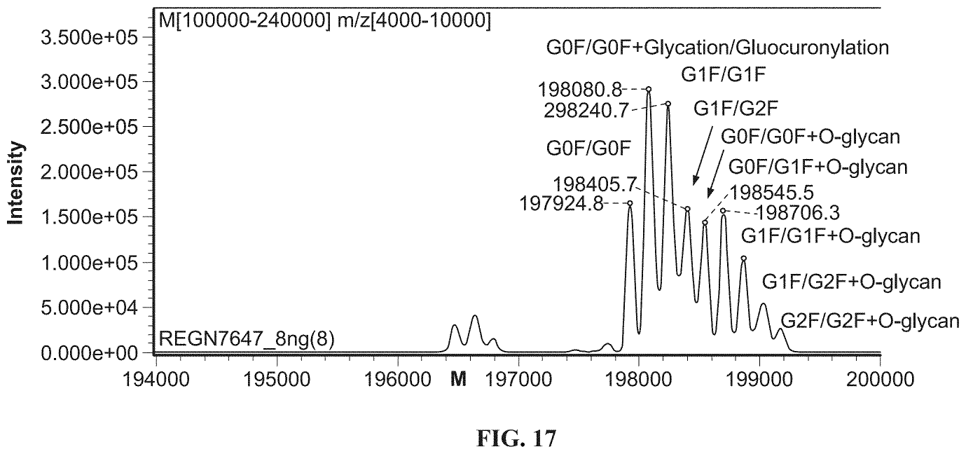

The basic variants were attributed mainly to one or two unprocessed C-terminal Lys residues and N-terminal pyroglutamate formation. In the acidic variants, in addition to commonly observed PTMs such as deamidation and glycation, mass differences matching O-glycosylation were also detected, as shown in FIG. 17. The O-glycan has a structure of Xyl+Gal+Gal+GlcA, which is a common O-glycan in mAb formats using G4S linkers, as reported in previous publications (Haberger et al.; Spahr et al., 2014, MAbs, 6(4):904-914; Spencer et al., 2013, J. Pharm. Sci, 102(11):3920-3924). The presence of O-glycan was confirmed by reduced peptide mapping. In summary, nCE-MS is suitable for the analysis of new formats and can reveal PTMs unique to common mAbs such as O-glycosylation.

In this study, characterization of charge variants and impurities of therapeutic antibodies was demonstrated with nCE-MS method, which provides high resolution separation and high sensitivity detection, with excellent system robustness. The electropherograms provided by nCE-MS analysis were similar to the chromatograms from SCX and the electropherograms from iCIEF. Therefore, this method can be used for peak identification in SCX and iCIEF studies. Antibodies with close pI values can be separated well. Co-migrated antibodies were identified individually based on simplified native mass spectra. Increased levels of acidic variants and Fab fragments resulting from incubation under stressed conditions were localized within the F(ab′)2 and Fc domains by subunit analysis. Furthermore, higher resolution subunit analysis revealed an additional acid variant introduced from isomerization and increased half glycosylated species under stressed condition. Moreover, high sensitivity detection enables the detection of impurities including truncations in NISTmAb and homospecific mAb impurities in bsAb. In addition to common therapeutic antibodies, nCE-MS analysis can be applied to characterize new modalities including mAbs with or without Fab arms and to detect PTMs such as O-glycosylation. Thus, the nCE-MS analysis shows great potential in the development of biopharmaceuticals, and implementation of this analysis may accelerate the timeline for elucidating drug candidate quality attributes and assist in investigations in product manufacturing.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for characterizing a monospecific antibody in a mixture of a bispecific antibody and its monospecific antibody side products, comprising:
(a) separating a mixture of a bispecific antibody and its monospecific antibody side products by molecular weight and/or charge in one or more capillaries using capillary electrophoresis;
(b) eluting said separated antibody and antibody side products from said one or more capillaries; and
(c) determining the mass of said eluted antibody and antibody side products by mass spectrometry, thereby characterizing said monospecific antibody,
wherein said monospecific antibody is maintained in native conditions, and wherein said capillary electrophoresis is in an integrated microfluidic platform.

2. The method of claim 1, further comprising determining a relative or absolute amount of said monospecific antibody in said mixture.

3. The method of claim 1, wherein said mixture includes an internal standard.

4. The method of claim 1, wherein said one or more capillaries comprise a separation matrix.

5. The method of claim 4, wherein said separation matrix comprises a sieving matrix configured to separate proteins by molecular weight.

6. The method of claim 1, wherein said monospecific antibody is of isotype IgG1, IgG2, IgG3, IgG4, or mixed isotype.

7. The method of claim 1, further comprising characterizing a second monospecific antibody in said mixture.

8. The method of claim 1, wherein an injection volume in said one or more capillaries is between about 1 nL and about 10 nL.

9. The method of claim 8, wherein an injection volume in said one or more capillaries is about 1 nL.
